(19) United States
(12) Patent Application Publication
(10) Pub. No.: US 2005/0054620 A1
(43) Pub. Date: Mar. 10, 2005

(54) Paricalcitol as a chemotherapeutic agent

(76) Inventors: H. Phillip Koeffler, Los Angeles, CA (US); Takashi Kumagai, Tokyo (JP)

Correspondence Address: Cathryn Campbell;McDERMOTT, WILL & EMERY, 7th Floor, 4370 La Jolla Village Drive, San Diego, CA, 92122, US

(21) Appl. No.: 10/756,890

(22) Filed: Jan. 13, 2004

Related U.S. Application Data
(60) Provisional application No. 60/439,932, filed on Jan. 13, 2003.

(51) Int. Cl. A61K 31/59

(52) U.S. Cl. 514/167

(57) ABSTRACT

The invention provides methods of reducing the severity of a proliferative disorder. One method involves administering to an individual having the proliferative disorder an effective amount of paricalcitol, wherein the paricalcitol reduces cellular proliferation, with the proviso that the cancer is not prostate cancer or head and neck squamous cell carcinoma. Another method of reducing the severity of a proliferative disorder provided by the invention involves administering to an individual having the proliferative disorder an effective amount of paricalcitol and an anti-cancer agent, wherein the combination of paricalcitol and the anti-cancer agent reduces cell proliferation, with the proviso that the proliferative disorder is not prostate cancer or head and neck squamous cell carcinoma.

This application claims the benefit of the filing date of U.S. Provisional Application No. 60/439,932, filed Jan. 13, 2003, which is incorporated herein by reference.

### BACKGROUND OF THE INVENTION

This invention relates generally to cancer therapeutics and, more specifically, to the use of the vitamin D analog paricalcitol as a chemotherapeutic agent.

Cancer is one of the leading causes of death in the United States. Each year, more than half a million Americans die from cancer, and more than one million are newly diagnosed with the disease. In cancer, neoplastic cells escape from their normal growth regulatory mechanisms and proliferate in an uncontrolled fashion, leading to the development of a malignant tumor. Tumor cells can metastasize to secondary sites if treatment of the primary tumor is either not complete or not initiated before substantial progression of the disease. Early diagnosis and effective treatment of malignant tumors is therefore essential for survival.

The current methods for treating cancer include surgery, radiation therapy and chemotherapy. A major problem with each of these treatments is their lack of specificity for cancer cells and numerous side-effects. For instance, due to their toxicity to normal tissues, the amount of radiation or chemotherapeutic agent that can be safely used is often inadequate to kill all neoplastic cells. Even a few residual neoplastic cells can be lethal, as they can rapidly proliferate and metastasize to other sites. Unfortunately, the toxicity associated with radiation and chemotherapy is manifested by unpleasant side effects, including nausea and hair loss, that severely reduce the quality of life for the cancer patient undergoing these treatments. Clearly, a means of treating cancer with less side-effects is needed.

Thus, there exists a need for effective anti-cancer agents. The present invention satisfies this need and provides related advantages as well.

### SUMMARY OF THE INVENTION

The invention provides methods of reducing the severity of a proliferative disorder. One method involves administering to an individual having the proliferative disorder an effective amount of paricalcitol, wherein the paricalcitol reduces cellular proliferation. In an embodiment of the invention, the proliferative disorder is cancer, with the proviso that the cancer is not prostate cancer or head and neck squamous cell carcinoma. In another embodiment, the proliferative disorder is a myelodysplastic syndrome. Exemplary cancers that can be treated using the method include leukemias, such as acute myelocytic leukemia and acute lymphocytic leukemia; multiple myeloma; breast cancer, and colon cancer.

Another method of reducing the severity of a proliferative disorder provided by the invention involves administering to an individual having the proliferative disorder an effective amount of paricalcitol and an anti-cancer agent, wherein the combination of paricalcitol and the anti-cancer agent reduces cell proliferation. In an embodiment of the invention, the proliferative disorder is cancer. In particular embodiments, the method is used to treat an individual having leukemia, multiple myeloma, breast cancer or colon cancer. In another embodiment of the invention, the proliferative disorder is a myelodysplastic syndrome. Exemplary anti-cancer agents that can be used in the method include daunomycin, arsenic trioxide, adriamycin, PS341, dexamethasone, taxol, 5-fluoroceracil and methotrexate. In an embodiment, arsenic trioxide is used with paricalcitol to treat leukemia, such as acute myelocytic leukemia or acute lymphocytic leukemia. In another embodiment, dexamethasone is used with paricalcitol to treat multiple myeloma. In a further embodiment, daunomycin is used with paricalcitol to treat myeloid leukemia. In an additional embodiment, PS341 is used with paricalcitol to treat myeloma. In a further embodiment, taxol is used with paricalcitol to treat prostate cancer or breast cancer. In yet another embodiment, adriamycin is used with paricalcitol to treat breast cancer. In an embodiment, 5-fluoroceracil is used with paricalcitol to treat colon cancer. In a further embodiment, methotrexate is used with paricalcitol to treat colon cancer.

Further provided by the invention is a method of reducing cancer recurrence. The method involves administering to an individual in cancer remission an effective amount of paricalcitol, wherein the paricalcitol reduces cancer cell proliferation. In one embodiment of the invention, the treated individual is in remission from leukemia, such as acute myelocytic leukemia or acute lymphocytic leukemia. In further embodiments, the individual is in remission from multiple myeloma, breast cancer, or colon cancer.

The invention provides another method of reducing cancer recurrence. The method involves administering to an individual in cancer remission an effective amount of paricalcitol and an anti-cancer agent, wherein the combination of paricalcitol and the anti-cancer agent reduces cancer cell proliferation. In embodiments of the invention, the individual to be treated is in remission from a cancer selected from leukemia, multiple myeloma, breast cancer and colon cancer. In embodiments of the invention, the anti-cancer agent is selected from daunomycin, arsenic trioxide, adriamycin, PS341, dexamethasone, taxol, 5-fluoroceracil and methotrexate. In one embodiment, arsenic trioxide is used with paricalcitol to treat an individual in remission from leukemia, such as acute myelocytic leukemia or acute lymphocytic leukemia. In another embodiment, dexamethasone is used with paricalcitol to treat an individual in remission from multiple myeloma. In another embodiment, daunomycin is used with paricalcitol to treat an individual in remission from myeloid leukemia. In a further embodiment, PS341 is used with paricalcitol to treat an individual in remission from myeloma. In an additional embodiment, taxol is used with paricalcitol to treat an individual in remission from prostate cancer or breast cancer. In yet another embodiment, adriamycin is used with paricalcitol to treat an individual in remission from breast cancer. In an embodiment, 5-fluoroceracil is used with paricalcitol to treat an individual in remission from colon cancer. In a further embodiment, methotrexate is used with paricalcitol to treat an individual in remission from colon cancer.

### BRIEF DESCRIPTION OF THE DRAWINGS

FIG. 1 shows the dose-response effects of paricalcitol on clonal proliferation of human cancer cell lines. Results of the dose-response clonogenic assays are shown for the leukemia (HL-60, NB-4, THP-1), colon cancer (HT-29, SW837) and myeloma (NCI-H929) cell lines cultured with either paricalcitol or 1,25(OH)₂D₃. Colonies were counted after 14 days. Results represent the mean±standard deviation (SD) of three independent experiments with triplicate dishes.

FIG. 2 shows effects of paricalcitol on the HL-60 leukemia cell line. (A) Cell cycle analysis of HL-60 cells by flow cytometry. HL-60 cells were cultured with paricalcitol (10⁻⁷ M) for 72 hrs, harvested and stained with propidium iodine (PI). Control cells were treated with vehicle alone. Results represent the mean±SD of three independent experiments. (B) HL-60 or NB-4 cells were treated with either paricalcitol (10⁻⁷ M) or 1,25(OH)₂D₃ (10⁻⁷ M). Cell lysates were prepared after 72 hrs and analyzed by Western blot with a series of antibodies (p21WAF1, p27KIP1, PTEN). Control cells were treated with vehicle alone. The amounts of protein were normalized by comparison to levels of GAPDH. (C) HL-60 cells were cultured with either paricalcitol (10⁻⁷ M) or 1,25(OH)₂D₃ (10⁻⁷ M) for 96 hrs and analyzed for expression of CD14 using flow cytometry. Dashed line indicates negative control antibody; black area, CD14 antibody. CD14 positive cells are in the area shown by M2. (D) HL-60 cells were cultured with paricalcitol (10⁻⁷ M) for 120 hrs, fixed and stained with Wright-Giemza stain for morphological analysis. The left panel shows HL-60 control cells (original magnification ×400) and the right panel shows HL-60 cells following exposure to paricalcitol (original magnification ×400).

FIG. 3 shows effects of paricalcitol on the NCI-929 myeloma cell line. (A) Cell cycle analysis of NCI-H929 cells by flow cytometry. HCI-H929 cells were cultured with either paricalcitol (10⁻⁷M) or 1,25(OH)₂D₃ (10⁻⁷M) for 72 hrs, harvested and stained with propidium iodine (PI). Control cells were treated with vehicle alone. (B) Quantitative analysis of apoptosis of NCI-H929 cell line exposed to either paricalcitol (10⁻⁷M), or 1,25(OH)₂D₃ (10⁻⁷M) for 96 hrs and analyzed by TUNEL assay. Results represent the mean±SD of three independent experiments. (C) NCI-H929 cells were treated with either paricalcitol (10⁻⁷M) or 1,25(OH)₂D₃ (10⁻⁷M) and cell lysates were prepared after 72 hrs. Cell lysates were used for Western blot analysis and probed sequentially with antibodies to p27KIP1, Bcl-2 and Bax. Control cells were treated with vehicle alone. Amount of protein was normalized by comparison to the amount of GAPDH.

FIG. 4 shows effects of paricalcitol on colon cancer cell lines. (A) HT-29, SW837, SW480 and HCT116 colon cancer cells were treated for 96 hrs with either paricalcitol (10⁻⁷M), 1,25(OH)₂D₃ (10⁻⁷M) or diluant (control). Growth (% of control) was measured by MTT assay. Results represent the mean±SD of three independent experiments with triplicate dishes. (B) HT-29 cells were exposed to either paricalcitol (10⁻⁷M) or 1,25(OH)₂D₃(10⁻⁷M). Cell lysates were prepared after 72 hrs of culture and analyzed by Western blot. The Western blot was probed sequentially with antibodies for p27KIP1, p21WAF1, cyclin D1, c-myc and E-Cadherin. Control cells were treated with vehicle alone. The quantity of protein was normalized by comparison to the amount of GAPDH. (C) HT-29 and SW837 cells were cultured with either paricalcitol (10⁻⁷M) or 1,25(OH)₂D₃ (10⁻⁷M) for 72 hrs. Cell lysates were prepared and analyzed by Western blot which was probed sequentially with antibodies to COX-1 and COX-2. Control cells were treated with vehicle alone. The amount of protein was normalized by comparison to the quantity of GAPDH.

FIG. 5 shows effects of paricalcitol on the growth of HT-29 colon cancer cells growing as tumors in nude mice. HT-29 cells were bilaterally injected subcutaneously into nude mice, forming two tumors per mouse. The mice were divided randomly into control and experimental groups. Paricalcitol (100 ng/mouse) was administered intraperitoneously for 3 days a week in the experimental groups (Monday, Wednesday, Friday). (A) Tumor volumes were measured every week. The mean volume±SD of 10 tumors in each group is shown. Tumor volumes were significantly different between the experimental and control groups (p=0.03). (B) After 4 weeks of therapy, tumors were removed from each group and weighed. The tumor weights were significantly different in the two groups (p=0.0004).

FIG. 6 shows expression of vitamin D receptor (VDR) in cell lines, expression of 24-hydroxylase in response to paricalcitol, and the effect of paricalcitol in cells isolated from wild-type and VDR knock out mice. (A) Cell lysates of HT-29, SW837, SW480, SW620 and HCT116 colon cancer cells were harvested and VDR expression was measured by Western blot. The amount of protein was normalized by comparison to levels of GAPDH. (B) HT-29 colon cancer cells were treated with paricalcitol (10⁻⁷M) for 0, 6, 12 or 24 hrs and RNA was harvested. Expression of 24 hydroxylase mRNA was analysed by RT-PCR. The amounts of mRNA were normalized by comparison to 18S RNA. (C) Mononuclear cells extracted from spleens of either wild type or VDR knock-out mice were treated with paricalcitol (10⁻⁸M) for either 12 or 24 hrs, and RNA was harvested. Expression of 24 hydroxylase mRNA was analysed by RT-PCR. The amounts of mRNA were normalized by comparison to 18S RNA. (D) Colony formation by mononuclear bone marrow cells from VDR knock-out (VDR-KO) and wild type (WT) mice. Mononuclear cells were obtained from femoral bone marrow plugs and grown in methylcellulose culture media with either paricalcitol (10⁻⁸M) or diluant. Colonies were counted on day 10 of culture. The number of total colonies (average) were 87 (control) and 66 (paricalcitol 10⁻⁸M) in wild type mice, and 110 (control) and 122 (paricalcitol 10⁻⁸M) in VDR-KO mice. The percentage of macrophage, granulocyte and mixed granulocyte/macrophage colonies are shown. Triplicate wells for each mouse and a total of three KO and three WT mice were studied. G, granulocyte colonies; G/M mixed granulocyte/macrophage colonies; M, macrophage colonies.

FIG. 7 shows the anti-proliferative effects of paricalcitol in combination with other anti-cancer agents on various types of cancer cell lines. Cancer cell lines including myeloid leukemia cells (HL-60, NB-4, U937); myeloma cells (NCI-H929, RPMI8228, ARH-77); prostate cancer cells (LNCaP, PC-3, DU145); breast cancer cells (MCF-7, MDA-MB231); and colon cancer cells (HT-29) were treated with paricalcitol and/or another anti-cancer agents including (A) daunomycin; (B) arsenic trioxide; (C and G) adriamycin; (D) PS-341; (E) dexamethasone; (F and H) taxol; (I) 5-fluoroceracil (5FU); (J) methotrexate (MTX); and (K) NS398 (COX-2 inhibitor), at the indicated doses and MTT assays were performed after 4 days. Control cells were treated with vehicle alone. Results represent the mean±SD of three independent experiments with triplicate dishes. (L) HL-60 and NB-4 myeloid leukemia cells were treated with paricalcitol and/or arsenic trioxide at the indicated doses and colony assays were performed. After 14 days, numbers of colonies were counted. Results represent the mean±SD of three independent experiments with triplicate dishes. HL-60 (M) and NB-4 (N) myeloid leukemia cell lines were treated with paricalcitol (10⁻⁸ M for HL-60, 10⁻⁷ M for NB-4) and/or arsenic trioxide (8×10⁻⁸ M for HL-60, 6×10⁻⁷ M for NB-4). Control cells were treated with vehicle alone. Cell numbers were counted by trypan blue assay every day for 6 days. Results represent the mean±SD of three independent experiments with triplicate dishes. (O) Prostate (LNCaP, PC-3, DU145), breast (MCF-7), colon (HT-29), endometholial (Ishikawa, HEC59, HEClB) and lung (NCI-H125, NCI-H520) cancer cell lines were treated with paricalcitol (0.1 μm) and arsenic trioxide (1 μm). MTT assays were performed after 4 days.

FIG. 8 shows that paricalcitol combined with arsenic trioxide markedly enhanced monocytic differentiation of HL-60 and NB-4 myeloid leukemia cells with subsequently increasing apoptosis. HL-60 and NB-4 myeloid leukemia cell lines were treated with paricalcitol (10⁻⁸ M for HL-60, 10⁻⁷ M for NB-4) and/or arsenic trioxide (8×10⁻⁸ M for HL-60, 6×10⁻⁷ M for NB-4). Control cells were treated with vehicle alone. (A) After 3 days, CD14 was measured by flow cytometry. Results represent the mean±SD of three independent experiments with triplicate dishes. (B) After 3 days, monocytic differentiation was measured by NBT reduction. Results represent the mean±SD of three independent experiments with triplicate dishes. (C) After 4 days, cell cycle analysis of NB-4 cells was performed by flow cytometry. The percent of cells in sub-G1 population is indicated. (D) After 4 days, quantitative analysis of apoptosis of NB-4 cell line was analyzed by TUNEL assay. Percent of the TUNEL positive cells is represented as the mean±SD of three independent experiments.

FIG. 9 shows modulation of gene expression by paricalcitol and arsenic trioxide in myeloid leukemia cells. HL-60 and NB-4 myeloid leukemia cell lines were treated with paricalcitol (0.01 μM for HL-60, 0.1 μM for NB-4) and/or arsenic trioxide (0.8 μM for HL-60, 0.6 μM for NB-4). Control cells were treated with vehicle alone. (A) After treatment of 3 days, mRNA was extracted and expression of 24-hydroxylase was measured by RT-PCR using specific primers for the gene. Cell lysates were also made and used for Western blot which was probed with antibodies to C/EBPβ. (B) Cell lysate of HL-60 cells were made after 3 days and used for Western blot which was probed sequentially with antibodies to Bcl-2, BclX_{L} and Bax (C) Cell lysates of HL-60 cells were made after 1, 2 or 3 days and used for Western blot which was probed sequentially with antibodies to phosphorylated ERK. (D) After pretreatment either with or without 25 nM of PD98059 for 1 hour, HL-60 cells were treated with paricalcitol (0.01 μM) and/or arsenic trioxide (0.8 μM) for 2 days. Control cells were treated with vehicle alone. CD14 expression was measured by flow cytometry.

FIG. 10 shows that paricalcitol in combination with arsenic trioxide overcomes the block of differentiation by PML-RARα fusion protein. (A) NB-4 myeloid leukemia cell lines were treated with paricalcitol (0.1 μM) and/or arsenic trioxide (0.6 μM) for 3 days. Control cells were treated with vehicle alone. Cell lysates were made and used for Western blot which was probed sequentially with antibodies to RARA to detect the fusion protein PML-RARα. (B) U937 cells were stably transfected with either the control MT vector (U937-PMT) or the PML-RARα cDNA under the control of the Zn2⁺-inducible murine metallothionein 1 promoter (PR9). Cells were treated either with (+) or without (−) Zn for 2 days. Paricalcitol and/or arsenic trioxide of the indicated doses were added to the cells. Cell lysates were harvested and used for Western blot which was probed with antibodies to RARα to detect PML-RARα fusion protein (120 kb). (C) U937-PMT and PR9 cells were treated either with or without Zn and with paricalcitol, and/or arsenic trioxide as indicated for 3 days, and CD14 expression was measured by flow cytometry.

FIG. 11 shows that arsenic trioxide suppressed the activity of 24-hydroxylase enzyme in leukemia cells. HL-60 (A) and NB-4 (B) myeloid leukemia cell lines were treated with paricalcitol (0.01 μM for HL-60, 0.1 μM for NB-4) and/or arsenic trioxide (0.8 μM for HL-60, 0.6 μM for NB-4) for 3 days. Control cells were treated with vehicle alone. The levels of 24.25(OH)₂D₃ were measured by HPLC analysis.

FIG. 12 shows that paricalcitol in combination with dexamethasone profoundly decreased proliferation of myeloma cells in vitro. Myeloma cell line, NCI-H929 was treated with either paricalcitol (0.01 μM) and/or dexamethasone (0.01 μM) for 3 days; control cells were treated with vehicle alone. (A) Cell cycle analysis was performed by flow cytometry. (B) % of sub-G1 population was measured by flow cytometry (left). TUNEL assay was performed for the quantitative analysis of the apoptotic cells (right). (D) Cell lysates were harvested and used for Western blot which was probed sequentially with antibodies to Bcl-2 and p₂₇^{KIP1}.

### DETAILED DESCRIPTION OF THE INVENTION

Paricalcitol (19-nor-1,25, (OH)₂D₃) is a synthetic analog of vitamin D (1,25(OH)₂D₃) that is currently approved by the Federal Drug Administration (FDA) for the clinical treatment of secondary hyperparathyroidism. The advantage of paricalcitol over 1,25(OH)₂D₃ is that paricalcitol has less calcemic potential and therefore has fewer side effects than 1,25(OH)₂D₃ (Llach et al., Am. J. Kidney Dis. 32(Suppl.):S48-54 (1998); and Martin et al., J. Am. Soc. Nephrol. 9:1427-1432 (1998)). Less calcemic Vitamin D analogs other than paricalcitol have been synthesized as described, for example, in Abe et al., Endocrinology 129:832-837 (1991); Zhou et al., Blood 78:75-82 (1991); Jung et al., Leuk. Res.18:453-463 (1994); Anzano et al., Cancer Res. 54:1653-1656 (1994); Pakkala et al., Leuk. Res. 19:65-72 (1995); Koike et al., Cancer Res. 57:4545-4550 (1997); Kubota et al., Cancer Res. 58:3370-3375 (1998); Hisatake et al., Cancer Res. 59:4023-4029 (1999); and Hisatake et al., Blood 97:2427-2433 (2001).

Antiproliferative effects of paricalcitol on prostate cancer cells in vitro were described in Chen et al., Clin. Cancer Res.6:901-908 (2000). As is disclosed herein, the inventors have discovered that paricalcitol has anti-proliferation activity against distinct cancer cell types, for example, leukemia cells, myeloma cells and colon cancer cells. Also as disclosed herein, the inventors have discovered that paricalcitol is associated with cell cycle arrest, induction of differentiation and apoptosis as well as decrease levels of COX-2. Further disclosed is that combination of paricalcitol with other anti-cancer agents, such as arsenic trioxide and dexamethasone, has anti-proliferation activity in myeloid leukemia cells and other cancer cell types.

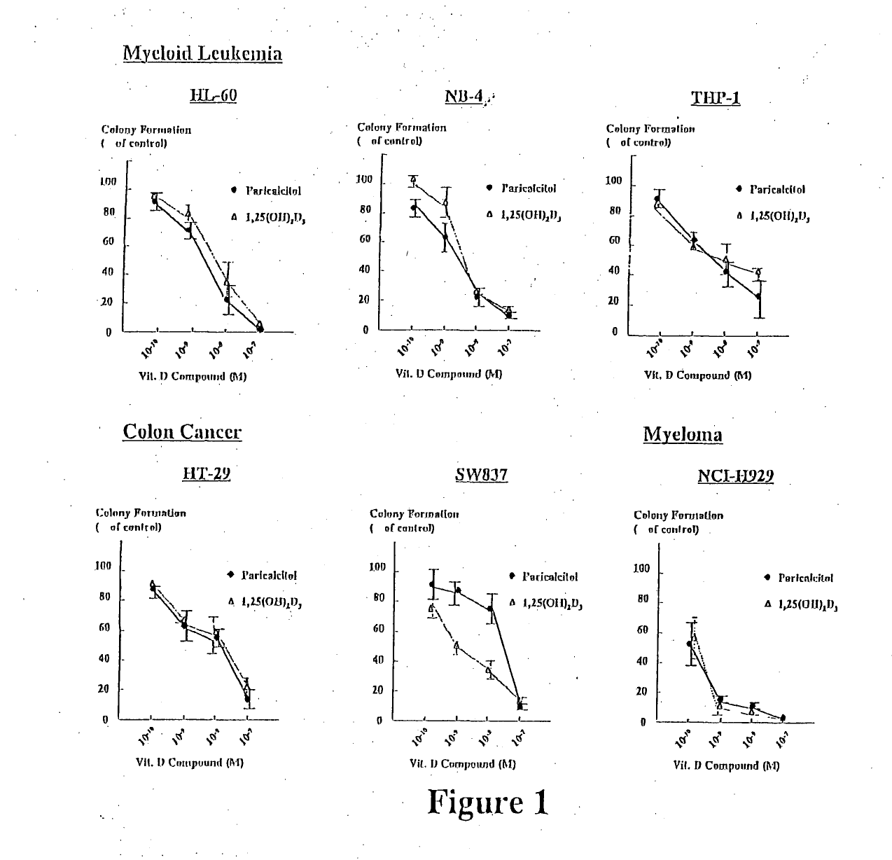

As disclosed herein, the anti-proliferation effects of paricalcitol on various human cancer cell lines including those from breast, lung, brain, myeloid leukemia, lymphoma, myeloma, colon and uterus was evaluated in vitro. The first screening used the rapid MTT assay with a 4 day exposure to paricalcitol. Cell lines that were sensitive to paricalitol using the MTT assay, which included myeloid leukemia cells (HL-60, NB-4, THP-1), myeloma cells (NCI-H929) and colon cancer cells (HT-29, SW837) were tested further. For example, as shown in FIG. 1, further antiproliferative studies of paricalcitol on the myeloid leukemia cell lines (HL-60, NB-4, THP-1), myeloma cells (NCI-H929) and the colon cancer cell lines (HT-29, SW837) in vitro was performed using the more sensitive soft agar colony assay. The concentration of paricalcitol that caused 50% inhibition (ED₅₀) of clonal growth was: HL-60, 2.4×10⁻⁹M; NB-4, 3.4×10⁻⁹M; THP-1, 5.8×10⁻⁹M; HT-29, 1.7×10⁻⁸M; SW837, 4.6×10⁻⁸M and NCI-H929, 2.0×10⁻¹¹M (see FIG. 1).

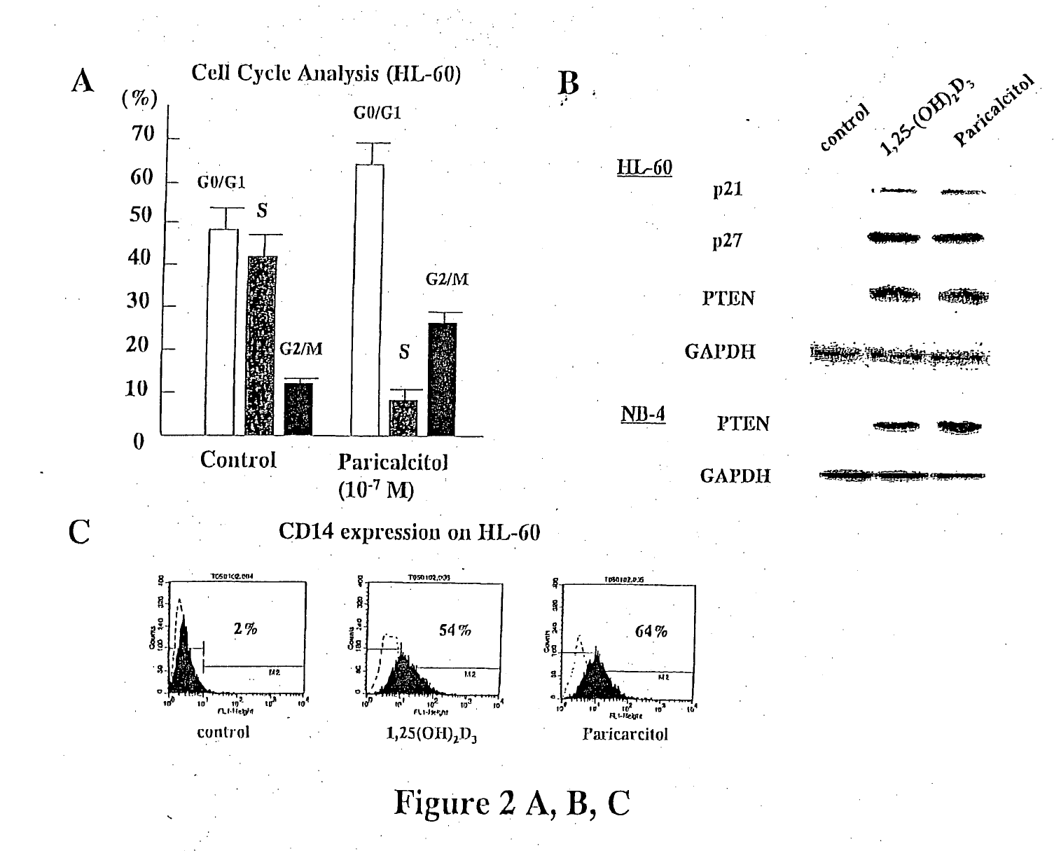

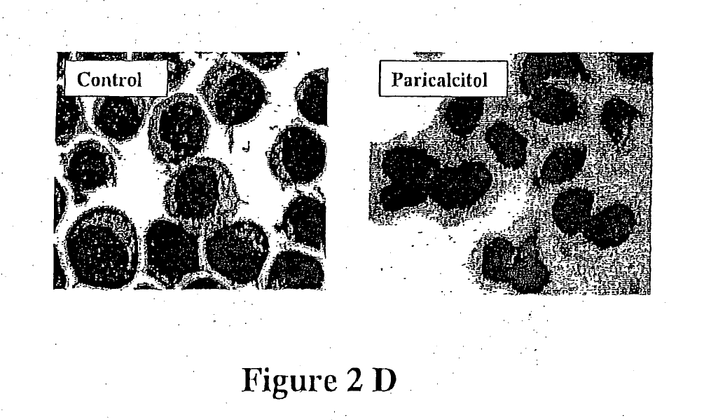

As further disclosed herein, paricalcitol has effects on the cell cycle status of myeloid leukemia cells in vitro. For example, as shown in FIG. 2A, cell cycle analysis of HL-60 cells after exposure to paricalcitol (10⁻⁷M, for 72 hr) showed an accumulation of cells in the G0/G1 phase (16% increase) and G2/M phase (17% increase), with a concomitant decrease in the proportion of cells in S phase (33% decrease). In addition, as shown in FIG. 2B, the expression of cyclin dependent kinase inhibitors (CDKIs) such as p21WAF1 and p27KIP1, which are associated with G0/G1 and G2/M accumulation of cells, was increased by exposure to both paricalcitol and 1,25(OH)₂D₃ (10⁻⁷M, for 72 hr). Both paricalcitol and 1,25(OH)₂D₃ induced p21WAF1 by approximately 7-fold and p27KIP1 by about 6-fold in the HL-60 cells (see FIG. 2B).

Induction of differentiation is sometimes useful as a less toxic cancer therapy that can supplement more aggressive approaches. This approach has been demonstrated in the use of all-trans-retinoic acid (ATRA) for the treatment of acute lymphocytic leukemia, which can induce complete remissions (Huang et al., Blood 72:567-572 (1988); Castaigne et al., Blood 76:1704-1709 (1990); and Warrell et al., New England J. Med. 324:1385-1393 (1991)).

As disclosed herein, paricalcitol has effects on the differentiation status of myeloid leukemia cells in vitro. For example, paricalcitol induced the expression of PTEN, a phosphatase that targets activated PI3 kinase and is associated with an anti-proliferative, pro-differentiation effect. As shown in FIG. 2B, paricalcitol (10⁻⁷M, for 72 hr) induced PTEN by 7-fold in HL-60 cells and by 25-fold in NB-4 myeloid leukemia cells. In addition, paricalcitol induced monocyte/macrophage-like differentiation of HL-60 cells as measured by induction of expression of the cell surface marker CD14. As shown in FIG. 2C, paricalcitol (10⁻⁷M, for 96 hrs) induced 65% of HL-60 cells to express CD14 and 1,25(OH)₂D₃ (10⁻⁷M, for 96 hrs) induced differentiation in 54% of HL-60 cells. Further, as shown in FIG. 2D, morphological examination showed monocytic differentiation of HL-60 cells treated with paricalcitol (10⁻⁷M, for 120 hrs). HL-60 control cells are large with round or oval nuclei, prominent nucleoli, and amphophilic cytoplasm. However, following exposure to paricalcitol, HL-60 cells developed monocytoid differentiation with oval, irregular, or indented nuclei, and abundant vacuolated cytoplasm (FIG. 2D).

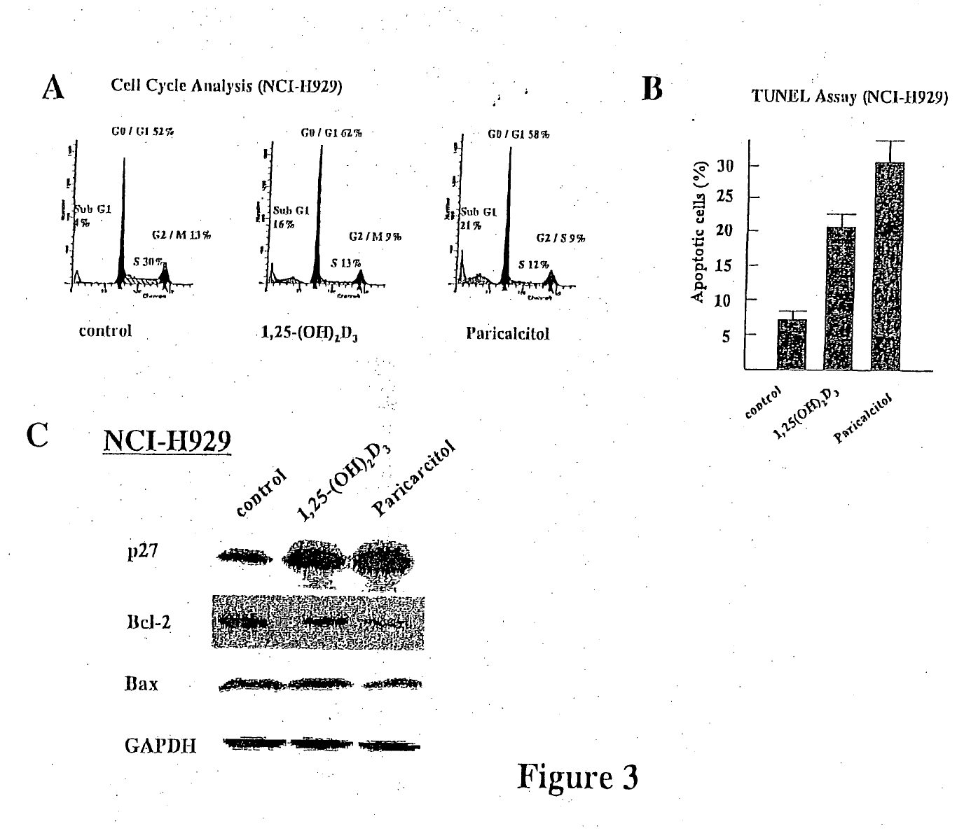

As disclosed herein, paricalcitol has anti-proliferative effects on human myeloma cells in vitro. For example, paricalcitol had an antiproliferative activity on NCI-H929 myeloma cells in a dose-dependent manner. The effect of paricalcitol on RPMI-8226 and ARH-77 myeloma cells was somewhat less than the effect on NCI-H929 cells. As shown in FIG. 3A, paricalcitol (10⁻⁷M, for 72 hrs) caused a 7% increase of accumulation in the G1/G0 phase and a 21% increase in the apoptotic, sub-G1 population of NCI-H929 cells, while these cells treated with diluant control had only a 4% sub-G1 population. Also as shown in FIG. 3A, 1,25(OH)₂D₃ (10⁻⁷M, for 72 hrs) caused accumulation of the cells in the G1/G0 phase (7% increase compared to control) and increased the sub-G1 cell population by 16%. In addition, as shown in the TUNEL assay in FIG. 3B, treatment of NCI-H929 cells with paricalcitol (10⁻⁷M, for 96 hrs) significantly increased apoptosis of NCI-H929 cells (31%) compared with diluant control cells (4%) (p<0.01). In the same assay, 1,25(OH)₂ D₃ treatment resulted in 20% apoptotic cells (see FIG. 3B). Also, under the same conditions ((10⁻⁷M, for 96 hrs), paricalcitol and 1,25(OH)₂D₃ both increased expression of p27KIP1 about 3-fold (see FIG. 3C), but had little effect on levels of p21WAF1. Further, as shown in FIG. 3C, paricalcitol and 1,25(OH)₂D₃ treatment (10⁻⁷M, for 72 hrs) decreased protein levels of the anti-apoptotic gene Bcl-2 by about 40%, but did not affect levels of the pro-apoptotic protein Bax.

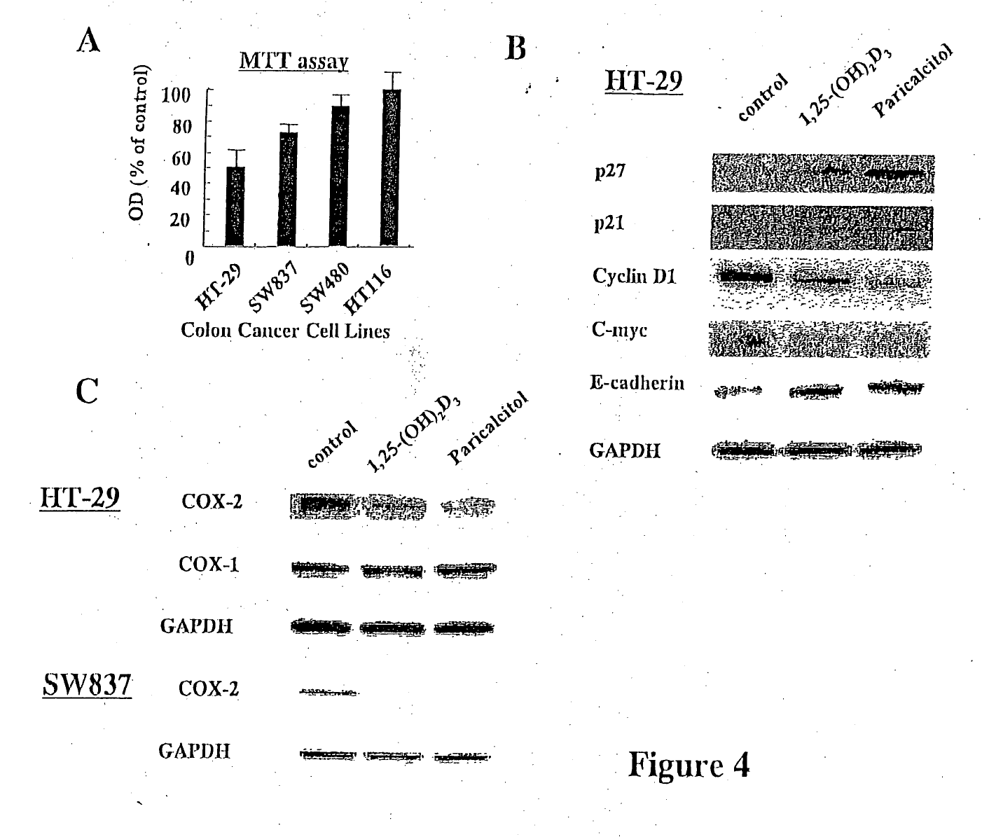

As disclosed herein, paricalcitol has anti-proliferative effects on colon cancer cells in vitro and in vivo. For example, as shown in the MTT assay in FIG. 4A, HT-29 and SW837 colon cancer cell lines were sensitive to treatment with paricalcitol (10⁻⁷M, for 96 hrs). Also as shown in FIG. 4A, the SW480 and HCT116 colon cancer cell lines were either only slightly sensitive or resistant to treatment with paricalcitol (10⁻⁷M, for 96 hrs). As shown in FIG. 4B, in HT-29 cells protein levels of p21WAF1 and p27KIP1 increased about 5-fold and 6-fold, respectively, after exposure to paricalcitol (10⁻⁷M, 72 hrs). At the same time, expression of cyclin D1 and c-myc decreased about 50% and 40%, respectively, after culture with either paricalcitol or 1,25(OH)₂D₃ (see FIG. 4B).

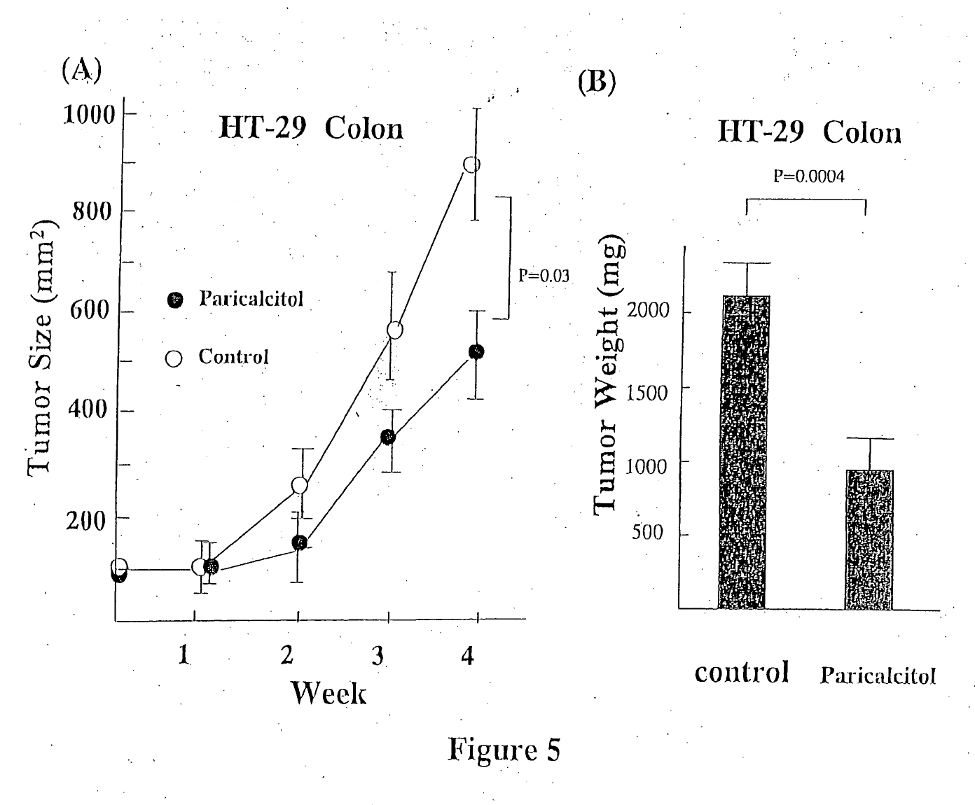

As disclosed herein, paricalcitol treatment can reduce tumor size and weight in vivo. The in vivo effect of paricalcitol on HT-29 human colon cancer tumors growing in nude mice was evaluated. Paricalcitol was injected intraperitoneously 3 days per week. Tumor volumes were measured weekly, and all mice were euthanized on the 5th week. Tumors were then dissected and weighed. As shown in FIGS. 5A and 5B, paricalcitol significantly suppressed both the growth of colon cancer tumors (p=0.03) (FIG. 5A) as well as their mean tumor weights compared to those growing in the diluant control mice (p=0.0004) (FIG. 5B). Serum calcium levels were 9.5±0.4 mg/dl in control mice and 10.2±0.7 in experimental mice. The serum calcium levels between the control and experimental mice were not significantly different (p=0.17), and each level was within the normal range.

As is further disclosed herein, paricalcitol when combined with arsenic trioxide showed an enhanced anti-proliferative effect against the myeloid leukemia cell lines, HL-60 and NB-4 as measured by MTT and colony assays compared to either drug alone. Paricalcitol (0.01 μM) alone induced monocytic differentiation of HL-60, while arsenic trioxide (0.8 μM) had little effect on differentiation, and when combined, the two drugs markedly enhanced monocytic differentiation of HL-60 as shown by NBT assay and induction of CD14 expression. The drug combination accumulated more HL-60 cells in G0/G1 cell cycle arrest and down-regulated Bcl-2 and Bcl-XL compared with treatment with either drug alone. Neither paricalcitol (0.1 μM) nor arsenic trioxide (0.6 μM) induced differentiation of NB-4 APL cells, but the combination caused monocytic differentiation and subsequently marked apoptosis.

Inhibition of cell proliferation by the combination of paricalcitol and arsenic trioxide and other anti-cancer agents is disclosed herein in Example VII. Inhibition of myeloma cell proliferation by the combination of paricalcitol and dexamethasone and other anti-cancer agents is disclosed herein in Example XI.

Based on the results disclosed herein, the invention provides methods of reducing the severity of a proliferative disorder. One method involves administering to an individual having the proliferative disorder an effective amount of paricalcitol, wherein the paricalcitol reduces cellular proliferation. In an embodiment of the invention, the proliferative disorder is cancer, with the proviso that the cancer is not prostate cancer or head and neck squamous cell carcinoma. In another embodiment, the proliferative disorder is a myelodysplastic syndrome. Exemplary cancers that can be treated using the method include leukemias, such as acute myelocytic leukemia and acute lymphocytic leukemia; multiple myeloma; breast cancer, and colon cancer.

Another method of reducing the severity of a proliferative disorder involves administering to an individual having the proliferative disorder an effective amount of paricalcitol and an anti-cancer agent, wherein the combination of paricalcitol and the anti-cancer agent reduces cell proliferation. In an embodiment of the invention, the proliferative disorder is cancer. In particular embodiments, the individual to be treated has leukemia, multiple myeloma, breast cancer or colon cancer. In another embodiment of the invention, the proliferative disorder is a myelodysplastic syndrome. Exemplary anti-cancer agents that can be used in the method include daunomycin, arsenic trioxide, adriamycin, PS341, dexamethasone, taxol, 5-fluoroceracil and methotrexate. In an embodiment, arsenic trioxide is used with paricalcitol to treat leukemia, such as acute myelocytic leukemia or acute lymphocytic leukemia. In an embodiment, dexamethasone is used with paricalcitol to treat multiple myeloma. In another embodiment, daunomycin is used with paricalcitol to treat myeloid leukemia. In a further embodiment, PS341 is used with paricalcitol to treat myeloma. In an additional embodiment, taxol is used with paricalcitol to treat prostate cancer or breast cancer. In yet another embodiment, adriamycin is used with paricalcitol to treat breast cancer. In an embodiment, 5-fluoroceracil is used with paricalcitol to treat colon cancer. In a further embodiment, methotrexate is used with paricalcitol to treat colon cancer.

As is described above, the invention provides methods for reducing the severity of a proliferative disorder. As used herein, the term “reducing the severity” means an arrest or decrease in clinical symptoms, physiological indicators or biochemical markers of proliferative disease. Clinical symptoms include perceptible, outward or visible signs of disease. Physiological indicators include detection of the presence or absence of physical and chemical factors associated with a process or function of the body. Biochemical markers include those signs of disease that are observable at the molecular level, such as the presence of a disease marker, such as a tumor marker. A tumor marker is a substance in the body that usually indicates the presence of cancer. Tumor markers are usually specific to certain types of cancer and are usually found in the blood or other tissue samples. One skilled in the art will be able to recognize specific clinical symptoms, physiological indicators and biochemical markers associated with a particular proliferative disease.

As is disclosed herein, paricalcitol or paricalcitol and an anti-cancer agent can be used to treat an individual having a proliferative disorder. Proliferative disorders include those diseases or abnormal conditions that result in unwanted or abnormal cell growth, viability or proliferation. Proliferative disorders include diseases such as cancer, in which the cells are neoplastically transformed, and diseases resulting from overgrowth of normal cells. For example, cell proliferative disorders include diseases associated with the overgrowth of connective tissues, such as various fibrotic diseases, including scleroderma, arthritis, alcoholic liver cirrhosis, keloid, and hypertropic scarring; vascular proliferative disorders, such as atherosclerosis; benign tumors, and the abnormal proliferation of cells mediating autoimmune disease. Those skilled in the art will be able to assess the severity of disease in an individual using an appropriate method for detecting and assessing the severity of a specific hyperproliferative disease.

By specific mention of the above categories of proliferative disorders, those skilled in the art will understand that such terms include all classes and types of these proliferative disorders. As used herein, the term “cancer” means a class of diseases characterized by the uncontrolled growth of aberrant cells, including all known cancers, and neoplastic conditions, whether characterized as malignant, benign, soft tissue or solid tumor. By exemplification, a list of known cancers is provided below in Table 1.

TABLE 1

HEMATOPORETIC NEOPLASMS
Lymphoid Neoplasms
Myeloid Neoplasms
Histiocytoses
Precursor B lymphoblastic leukemia/lymphoma (ALL)
Precursor T lymphoblastic leukemia/lymphoma (ALL)
Chronic lymphocytic leukemia/small lymphocytic
lymphoma (SLL)
Lymphoplasmacytic lymphoma
Mantle cell lymphoma
Follicular lymphoma
Marginal zone lymphoma
Hairy cell leukemia
Plasmacytoma/plasma cell myeloma
Diffuse large B-cell lymphoma
Burkitt lymphoma
T-cell chronic lymphocytic leukemia
Large granular lymphocytic leukemia
Mycosis fungoids and sezary syndrome
Peripheral T-cell lymphoma, unspecified
Angioimmunoblastic T-cell lymphoma
Angiocentric lymphoma (NK/T-cell lymphoma)
Intestinal T-cell lymphoma
Adult T-cell leukemia/lymphoma
Anaplastic large cell lymphoma
Hodgkin Diseases (HD)
Acute myclogenous leukemia (AML)
Myclodysplastic syndromes
Chronic Myclofroliferative Disorders
Chronic Myclogenous Leukemia (CML)
Polycythemia Vera
Essential Thrombocytosis
Myelofibrosis with Myeloid Metaplasia
Hemangioma
Lymphangioma
Glomangioma
Kaposi Sarcoma
Hemanioendothelioma
Angiosarcoma
Hemangiopericytoma
HEAD & NECK
Basal Cell Carcinoma
Squamous Cell Carcinoma
Ceruminoma
Osteoma
Nonchromaffin Paraganglioma
Acoustic Neurinoma
Adenoid Cystic Carcinoma
Mucoepidermoid Carcinoma
Malignant Mixed Tumors
Adenocarcinoma
Lymphoma
Fibrosarcoma
Osteosarcoma
Chondrosarcoma
Melanoma
Olfactory Neuroblastoma
Isolated Plasmocytoma
Inverted Papillomas
Undifferentiated Carcinoma
Mucoepidermoid Carcinoma
Acinic Cell Carcinoma
Malignant Mixed Tumor
Other Carcinomas
Amenoblastoma
Odontoma
THYMUS
Malignant Thymoma
Type I (Invasive thymoma)
Type II (Thymic carcinoma)
Squamous cell carcinoma
Lymph epithelioma
LUNG
Squamous Cell Carcinoma
Adenocarcinoma
Bronchial derived
Acinar; papillary; solid
Bronchioalveolar
Small Cell Carcinoma
Oat Cell
Intermediate Cell
Large Cell Carcinoma
Undifferentiated; giant cell; clear cell
Malignant Mesothelioma
Sarcomotoid Type
Epithelial Type
GASTROINTESTINAL TRACT
Squamous Cell Carcinoma
Adenocarcinoma
Carcinoid
Malignant Melanoma
Adenocarcinoma
Gastric Carcinoma
Gastric Lymphoma
Gastric Stromal Cell Tumors
Lymphoma
Kaposi's Sarcoma
Intestinal Stromal Cell Tumors
Carcinids
Malignant Mesethelioma
Non-mucin producing adenocarcinoma
LIVER AND THE BILIARY TRACT
Hepatocellular Carcinoma
Cholangiocarcinoma
Hepatoblastoma
Angiosarcoma
Fibrolameller Carcinoma
Carcinoma of the Gallbladder
Adenocarcinoma
Squamous Cell Carcinoma
Papillary, poorly differentiated
PANCREAS
Adenocarcinoma
Cystadenocarcinoma
Insulinoma
Gastrinoma
Glucagonamoa
KIDNEY
Renal Cell Carcinoma
Nephroblastoma (Wilm's Tumor)
LOWER URINARY TRACT
Urothelial Tumors
Squamous Cell Carcinoma
Mixed Carcinoma
Adenocarcinoma
Small Cell Carcinoma
Sarcoma
MALE GENITAL TRACT
Squamous Cell Carcinoma
Sarcinoma
Speretocytic Sarcinoma
Embyonal Carcinoma
Choriocarcinoma
Teratoma
Leydig Cell Tumor
Sertoli Cell Tumor
Lymphoma
Adenocarcinoma
Undifferentiated Prostatic Carcinoma
Ductal Transitional Carcinoma
FEMALE GENITAL TRACT
Squamous Cell Carcinoma
Basal Cell Carcinoma
Melanoma
Fibrosarcoma
Intaepithelial Carcinoma
Adenocarcinoma Embryonal Rhabdomysarcoma
Large Cell Carcinoma
Neuroendocrine or Oat Cell Carcinoma
Adenocarcinoma
Adenosquamous Carcinoma
Undifferentiated Carcinoma
Carcinoma
Adenoacanthoma
Sarcoma
Carcinosarcoma
Leiomyosarcoma
Endometrial Stromal Sarcoma
Serous Cystadenocarcinoma
Mucinous Cystadenocarcinoma
Endometrioid Tumors
Adenosarcoma
Celioblastoma (Brenner Tumor)
Clear Cell Carcinoma
Unclassified Carcinoma
Granulosa-Theca Cell Tumor
Sertoli-Leydig Cell Tumor
Disgerminoma
Teratoma
BREAST
Phyllodes Tumor
Sarcoma
Paget's Disease
Carcinoma
Insitu Carcinoma
Invasive Carcinoma
ENDOCRINE SYSTEM
Adenoma
Carcinoma
Meningnoma
Cramiopharlingioma
Papillary Carcinoma
Follicular Carcinoma
Medullary Carcinoma
Anoplastic Carcinoma
Adenoma
Carcinoma
Pheochromocytoma
Neuroblastome
Paraganglioma
Pineal
Pineoblastoma
Pineocytoma
SKIN
Melanoma
Squamous cell carcinoma
Basal cell carcinoma
Merkel cell carcinoma
Extramamary Paget's Disease
Paget's Disease of the nipple
Kaposi's Sarcoma
Cutaneous T-cell lymphoma
BONES, JOINTS, AND SOFT TISSUE TUMORS
Multiple Myeloma
Malignant Lymphoma
Chondrosacrcoma
Mesenchymal Chondrosarcoma
Osteosarcoma
Ewing Tumor (Ewing Sarcoma)
Malignant Giant Cell Tumor
Adamantinoma
Malignant Fibrous Histiocytoma
Desmoplastc Fibroma
Fibrosarcoma
Chordoma
Hemangioendothelioma
Memangispericytoma
Liposarcoma
Malignant Fibrous Histiocytoma
Rhabdomysarcoms
Leiomyosarcoma
Angiosarcoma
NERVOUS SYSTEM
Schwannoma
Neurofibroma
Malignant Periferal Nerve Sheath Tumor
Astrocytoma
Fibrillary Astrocytoma
Glioblastoma Multiforme
Brain Stem Glioma
Pilocytic Astrocytoma
Pleomorphic Xanthorstrocytoma
Oligodendroglioma
Ependymoma
Gangliocytoma
Cerebral Neuroblastoma
Central Neurocytoma
Dysembryoplastic Neuroepithelial Tumor
Medulloblastoma
Malignant Meningioma
Primary Brain Lymphoma
Primary Brain Germ Cell Tumor
EYE
Carcinoma
Squamous Cell Carcinoma
Mucoepidermoid Carcinoma
Melanoma
Retinoblastoma
Glioma
Meningioma
HEART
Myxoma
Fibroma
Lipoma
Papillary Fibroelastoma
Rhasdoyoma
Angiosarcoma
Other Sarcoma
HISTIOCYTOSES
Langerhans Cell Histiocytosis

The methods of the invention for reducing the severity of a proliferative disorder can be used to treat a variety of premalignant conditions. As used herein, the term “premalignant” means a precancerous state of a tissue having a an abnormality in which cancer is more likely to occur than in a normal tissue of the same type. Such an abnormality can be characterized based on histological abnormalities of cytology and/or architecture or biochemical differences between the precancerous versus normal states of the tissue. Particular differences depend on the particular type of tissue undergoing a premalignant process and are described in the art, for example, as metaplasia, dysplasia, hyperplasia, carcinoma in situ, angiogenic and the like, depending on the degree of structural and/or functional change compared to normal.

In one embodiment, paricalcitol or paricalcitol and an anti-cancer agent are used to treat an individual having a myelodysplastic syndrome. The myelodysplastic syndromes (MDS) are a group of disorders characterized by one or more peripheral blood cytopenias secondary to bone marrow dysfunction. The syndromes can arise de novo, or secondarily after treatment with chemotherapy and/or radiation therapy for other diseases. The myelodysplastic syndromes (MDS) are classified according to features of cellular morphology, etiology, and clinical presentation. The morphological classification of the MDS is generally based largely on the percent of myeloblasts in the bone marrow and blood, the type and degree of myeloid dysplasia, and the presence of ringed sideroblasts (Bennett et al., Br J Haematol 51 (2):189-99 (1982)). The clinical classification of the MDS generally depends upon whether or not there is an identifiable etiology and whether or not the MDS has been treated previously. Examples of MDS include, but are not limited to, refractory anemia (RA), refractory cytopenia with multilineage dysplasia (RCMD), refractory anemia with ringed sideroblasts (RARS, refractory anemia with excess blasts (RAEB), myelodysplastic syndrome, unclassifiable (MDS-U), myelodysplastic syndrome associated with del(5q), AML with multilineage dysplasia following a myelodysplastic syndrome, and myelodysplastic/myeloproliferative diseases (MDS/MPD).

The methods for reducing the severity of a proliferative disorder can be used to treat cancer. In embodiments of the invention, paricalcitol or paricalcitol and an anti-cancer agent are used to treat an individual having leukemia. Leukemia is a malignant neoplasm of blood-forming tissues, and is characterized by abnormal proliferation of leukocytes. Leukemias are generally classified according to cellular maturity. Acute leukemias consist of predominantly immature cells (usually blast forms) while chronic leukemias consist of predominantly more mature cells.

Acute leukemias are divided into lymphoblastic (ALL) and myelogenous (AML) types, which may be further subdivided by morphologic and cytochemical appearance, for example, according to the French-American-British (FAB) classification or immunophenotype. The specific B-cell and T-cell and myeloid-antigen monoclonal antibodies, together with flow cytometry, are useful for classifying ALL versus AML. Chronic leukemias are described as lymphocytic (CLL) or myelocytic (CML). Myelodysplastic syndromes represent progressive bone marrow failure but with an insufficient proportion of blast cells (<30%) for definite diagnosis of AML; 40 to 60% of cases evolve into AML.

Acute lymphocytic leukemia (ALL) results from an acquired genetic injury to the DNA of a single cell in the bone marrow. The disease is often referred to as acute lymphoblastic leukemia because the leukemic cell that replaces the normal marrow is the leukemic lymphoblast. The effects of ALL include uncontrolled and exaggerated growth and accumulation of cells called “lymphoblasts” or “leukemic blasts,” which fail to function as normal blood cells and blockade of the production of normal marrow cells, leading to a deficiency of red cells (anemia), platelets (thrombocytopenia), and normal white cells (especially neutrophils) in the blood.

Acute myeloid leukemia (AML) results from acquired genetic damage to the DNA of developing cells in the bone marrow. The effects of AML include uncontrolled, exaggerated growth and accumulation of cells called “leukemic blasts” that fail to function as normal blood cells and blockade of the production of normal marrow cells, leading to a deficiency of red cells (anemia), and platelets (thrombocytopenia) and normal white cells (especially neutrophils) in the blood.

The methods of the invention for reducing the severity of a proliferation disorder or reducing cancer recurrence can be used for a variety of leukemias, including the specific leukemias described above.

In embodiments of the invention, paricalcitol or paricalcitol and an anti-cancer agent are used to treat an individual having multiple myeloma. Multiple myeloma is a systemic malignancy of plasma cells. Generally, myeloma is referred to by the type of immunoglobulin or light chain (kappa or lambda type) produced by the cancerous plasma cell. The frequency of the various immunoglobulin types of myeloma parallels the normal serum concentrations of the immunoglobulins. The most common myeloma types are IgG and IgA. IgG myeloma accounts for about 60% to 70% of all cases of myeloma and IgA accounts for about 20% of cases. Few cases of IgD and IgE myeloma have been reported. Although a high level of M protein in the blood is a hallmark of myeloma disease, about 15% to 20% of patients with myeloma produce incomplete immunoglobulins, containing only the light chain portion of the immunoglobulin (light chain myeloma). A rare form of myeloma called nonsecretory myeloma affects about 1% of myeloma patients. In this form of the disease, plasma cells do not produce M protein or light chains. The methods of the invention for reducing the severity of a proliferation disorder or reducing cancer recurrence are application to any type of myeloma.

The methods of the invention for reducing the severity of a proliferative disorder and for reducing cancer recurrence involve administering an effective amount of paricalcitol, or an effective amount of paricalcitol and an anti-cancer agent. As used herein, the term “effective amount” when used in reference to reducing the severity of a proliferative disease, such as cancer, means an amount of paricalcitol or paricalcitol and an anti-cancer agent administered to an individual required to effect a decrease in the extent, amount or rate of spread of a neoplastic condition or pathology. When used in reference to reducing cancer recurrence, the term means an amount of paricalcitol or paricalcitol and an anti-cancer agent administered to an individual required to reduce cancer recurrence or risk of cancer recurrence. The amount of a paricalcitol and an anti-cancer agent required to be effective will depend, for example, on the type of anti-cancer agent administered and the pathological condition to be treated, as well as the weight and physiological condition of the individual, and previous or concurrent therapies. An amount considered as an effective amount for a particular application of paricalcitol or paricalcitol and an anti-cancer agent will be known or can be determined by those skilled in the art, using the teachings and guidance provided herein. One skilled in the art will recognize that the condition of the patient can be monitored throughout the course of therapy and that the amount of the modulating compound that is administered can be adjusted according to the individual's response to therapy.

In the methods of the invention for reducing the severity of a proliferative disorder and for reducing cancer recurrence, administration of paricalcitol or the combination of paricalcitol and an anti-cancer agent reduces cellular proliferation. As used herein, the term “reduces” when used in reference to cellular proliferation means effecting a decrease in the extent, amount or rate of cell growth.

As is disclosed herein in Examples I to V, VII and XI, paricalcitol or a combination of paricalitol with one or more anti-cancer agents reduces cancer cell proliferation. Therefore, administration of paricalcitol or a combination of paricalcitol with one or more anti-cancer agents can be used to reduce cancer cell proliferation in order to reduce cancer recurrence in an individual in cancer remission. Accordingly, the invention provides a method of reducing cancer recurrence. The method involves administering to an individual in cancer remission an effective amount of paricalcitol, wherein the paricalcitol reduces cancer cell proliferation. In one embodiment of the invention, the treated individual is in remission from leukemia, such as acute myelocytic leukemia or acute lymphocytic leukemia. In further embodiments, the individual is in remission from multiple myeloma, breast cancer, or colon cancer.

The invention provides another method of reducing cancer recurrence. The method involves administering to an individual in cancer remission an effective amount of paricalcitol and an anti-cancer agent, wherein the combination of paricalcitol and the anti-cancer agent reduces cancer cell proliferation. In embodiments of the invention, the individual to be treated is in remission from a cancer selected from leukemia, multiple myeloma, breast cancer and colon cancer. In embodiments of the invention, the anti-cancer agent is selected from daunomycin, arsenic trioxide, adriamycin, PS341, dexamethasone, taxol, 5-fluoroceracil and methotrexate. In one embodiment, arsenic trioxide is used with paricalcitol to treat an individual in remission from leukemia, such as acute myelocytic leukemia or acute lymphocytic leukemia. In another embodiment, dexamethasone is used with paricalcitol to treat an individual in remission from multiple myeloma. In another embodiment, daunomycin is used with paricalcitol to treat an individual in remission from myeloid leukemia. In a further embodiment, PS341 is used with paricalcitol to treat an individual in remission from myeloma. In an additional embodiment, taxol is used with paricalcitol to treat an individual in remission from prostate cancer or breast cancer. In yet another embodiment, adriamycin is used with paricalcitol to treat an individual in remission from breast cancer. In an embodiment, 5-fluoroceracil is used with paricalcitol to treat an individual in remission from colon cancer. In a further embodiment, methotrexate is used with paricalcitol to treat an individual in remission from colon cancer.

An individual in remission from cancer can be treated according to a method of the invention to reduce the risk of cancer recurrence. As used herein, the term “recurrence” means growth or neoplastic or cancerous cells after a tumor or other cancerous condition has been successfully treated, such as by surgical or chemically-induced removal or disintegration of cancerous cells. Such recurrence can involve dissemination of cancerous cells into local or distant tissues and organs with respect to the primary cancer.

For reducing the severity of a proliferative disorder or for reducing cancer recurrence, an effective amount can be, for example, between about 10 μg/kg to 500 mg/kg body weight, for example, between about 0.1 mg/kg to 100 mg/kg, or preferably between about 1 mg/kg to 50 mg/kg, depending on the treatment regimen. For example, if paricalcitol, an anti-cancer agent, or both, or formulation containing paricalcitol, an anti-cancer agent, or both is administered from one to several times a day, then a lower dose would be needed than if a formulation were administered weekly, or monthly or less frequently. Similarly, formulations that allow for timed-release of paricalcitol or paricalcitol and an anti-cancer agent, would provide for the continuous release of a smaller amount of paricalcitol or paricalcitol and an anti-cancer agent than would be administered as a single bolus dose. For example, paricalcitol or paricalcitol and an anti-cancer agent can be administered at between about 1-5 mg/kg/week. Studies employing paricalcitol administration are well known; exemplary regimes for paricalcitol administration are published in relation to the commercially available paricalcitol preparation ZEMPLAR™ (Abbott Laboratories, North Chicago, Ill.). For example, one recommended initial dose of ZEMPLAR™ is 0.04 mcg/kg to 0.1 mcg/kg (2.8-7 mcg) administered as a bolus dose no more frequently than every other day at any time during dialysis. Doses as high as 0.24 mcg/kg (16.8 mcg) have been safely administered.

Formulations of paricalcitol or paricalcitol and an anti-cancer agent also can be delivered in an alternating administrations so as to combine their antiproliferative effects over time. For example, paricalcitol or an anti-cancer agent or a combination thereof can be administered in a single bolus dose followed by multiple administrations of paricalcitol, the anti-cancer agent or combination thereof. Those skilled in the art will know or can determine a specific regime of administration which is effective for a particular application using the teachings and guidance provided herein together with diagnostic and clinical criteria known within the field of art of the particular proliferative disorder.

The dosage of paricalcitol or paricalcitol and an anti-cancer agent required to be therapeutically effective will depend, for example, on the pathological condition to be treated, the route and form of administration, the weight and condition of the individual, and previous or concurrent therapies. The appropriate amount considered to be an effective dose for a particular application of the method can be determined by those skilled in the art, using the guidance provided herein. For example, the amount can be extrapolated from in vitro or in vivo assays described herein. One skilled in the art will recognize that the condition of the patient can be monitored throughout the course of therapy and that the amount of the agent that is administered can be adjusted accordingly.

The methods of the invention for reducing the severity of a proliferative disorder or reducing cancer recurrence can be practiced in conjunction with other therapies. For example, the methods of the invention can be practiced prior to, during, or subsequent to conventional cancer treatments such as surgery, chemotherapy, including administration of cytokines and growth factors, radiation or other methods known in the art.

The methods of the invention for reducing the severity of a proliferative disorder do not include the use of paricalcitol with radiotherapy or brachytherapy for treating prostate cancer, as is described in Dunlap et al. British Journal of Cancer 89:746-753 (2003); nor do they include the use of paricalcitol together with cisplatin for treating head and neck squamous cell carcinomas, as has been described in Huang et al. Proc. Am. Sco. Clin. Oncol. 22:509 (2003); nor the use of paricalcitol for treating prostate cancer cells, as has been described in Chen et al. Clin Cancer Res 6(3):901-8 (2000).

As is shown herein in Examples VII and XI, for example, a combination of paricalcitol with an anti-cancer agent can act in a synergistic manner. Such synergistic activity, or even additive activity, can result in a reduction in tumor mass caused by the conventional therapy, increasing the effectiveness of paricalcitol or a combination of paricalcitol and an anti-cancer agent, and vice versa. Non-limiting examples of anti-cancer drugs that are suitable for co-administration are well known to those skilled in the art of cancer therapy and include an alkylating agent such as mechlorethamine, chlorambucil, cyclophosphamide, melphalan, ifosfamide; an antimetabolite such as methotrexate, 6-mercaptopurine, 5-fluorouracil or cytarabine; an antibody such as Rituxan, Herceptin, or MabThera; a plant alkaloid such as vinblastine or vincristine, or etoposide; an antibiotic such as doxorubicin, daunomycin, bleomycin, or mitomycin; a nitrosurea such as carmustine or lomustine; an inorganic ion such as cisplatin; a biological response modifier such as interferon; an enzyme such as aspariginase; or a hormone such as tamoxifen or flutamide. Other exemplary anti-cancer agents include aminoglutethimide, amsacrine (m-AMSA), azacitidine, asparaginase, bleomycin, busulfan, carboplatin, carmustine (BCNU), chlorambucil, cisplatin (cis-DDP), cyclophosphamide, cytarabine HCl, dacarbazine, dactinomycin, daunorubicin HCl, doxorubicin HCl, erythropoietin, estramustine phosphate sodium, etoposide (V16-213), floxuridine, fluorouracil (5-FU), flutamide, hexamethylmelamine (HMM), hydroxyurea (hydroxycarbamide), ifosfamide, interferon alpha, interleukin 2, leuprolide acetate (LHRH-releasing factor analogue), lomustine (CCNU), mechlorethamine HCl (nitrogen mustard), melphalan, mercaptopurine, mesna, methotrexate (MTX), mitoguazone (methyl-GAG, methyl glyoxal bis-guanylhydrazone, MGBG), mitomycin, mitotane (o. p′-DDD), mitoxantrone HCl, octreotide, pentostatin, plicamycin, procarbazine HCl, semustine (methyl-CCNU), streptozocin, tamoxifen citrate, teniposide (VM-26), thioguanine, thiotepa, vinblastine sulfate, vincristine sulfate, vindesine sulfate, Herceptin, and MabThera. These and other anti-cancer agents, are known in the art and formulations suitable for pharmaceutical use are known as described, for example, in The Merck Manual 16th Ed., Merck Res. Labs., Rahway N.J. (1992).

In the methods of the invention for reducing the severity of a proliferative disease or reducing cancer recurrence, paricalcitol or paricalcitol and an anti-cancer agent can be formulated together with a pharmaceutically acceptable carrier. Suitable pharmaceutically acceptable carriers are well known in the art and include, for example, aqueous or organic solvents such as physiologically buffered saline, glycols, glycerol, oils or injectable organic esters. A pharmaceutically acceptable carrier can also contain a physiologically acceptable agent that acts, for example, to stabilize or increase solubility of a pharmaceutical composition. Such a physiologically acceptable agent can be, for example, a carbohydrate such as glucose, sucrose or dextrans; an antioxidant such as ascorbic acid or glutathione; a chelating agent; a low molecular weight polypeptide; or another stabilizer or excipient. Pharmaceutically acceptable carriers including solvents, stabilizers, solubilizers and preservatives, are well known in the art as described, for example, in Martin, Remington's Pharm. Sci. 15th Ed. (Mack Publ. Co., Easton, 1975).

Appropriate distribution in vivo can be provided by rechargeable or biodegradable devices, particularly where concentration gradients or continuous delivery is desired. Various slow release polymeric devices are known in the art for the controlled delivery of drugs, and include both biodegradable and non-degradable polymers and hydrogels. Polymeric device inserts can allow for accurate dosing, reduced systemic absorption and in some cases, better patient compliance resulting from a reduced frequency of administration. Those skilled in the art understand that the choice of the pharmaceutical formulation and the appropriate preparation of the compound will depend on the intended use and mode of administration.

Suitable routes of administration of paricalcitol or paricalcitol and an anti-cancer agent include, but are not limited to, oral, topical, sublingual, intraocular, intradermal, parenteral, intranasal, intravenous, intramuscular, intraspinal, intracerebral and subcutaneous routes.

Paricalcitol or paricalcitol and an anti-cancer agent can be peripherally administered, without limitation, orally in any acceptable form such as in a tablet, pill, capsule, powder, liquid, suspension, emulsion or the like; as an aerosol; as a suppository; by intravenous, intraperitoneal, intramuscular, subcutaneous or parenteral injection; by transdermal diffusion or electrophoresis; topically in any acceptable form such as in drops, creams, gels or ointments; and by minipump or other implanted extended release device or formulation. It is understood that slow-release formulations can be useful in the methods of the invention. It is further understood that the frequency and duration of dosing will be dependent, in part, on the effect desired and the half-life of the active ingredients and that a variety of routes of administration are useful for delivering slow-release formulations, as detailed herein above.

Animal models of specific hyperproliferative diseases can be used to assess the efficacy of particular dosages, formulations or routes of administration of paricalcitol or paricalcitol and an anti-cancer agent. A variety of animal tumor models are known in the art that are predictive of the effects of therapeutic treatment. These models generally include the inoculation or implantation of a laboratory animal with heterologous tumor cells followed by simultaneous or subsequent administration of a therapeutic treatment. The efficacy of the treatment is determined by measuring the extent of tumor growth or metastasis. Measurement of clinical or physiological indicators can alternatively or additional be assessed as an indicator of treatment efficacy. Exemplary animal tumor models can be found described in, for example, Brugge et al. Origins of Human Cancer, Cold Spring Harbor Laboratory Press, Plain View, New York, (1991).

The methods of the invention involve administering paricalcitol. Paricalcitol can be obtained as a commercial preparation (ZEMPLAR; Abbott Laboratories, North Chicago, Ill.) or can be prepared synthetically. Procedures for preparing paricalcitol and similar compounds are generally described, for example, in U.S. Pat. No. 5,976,784.

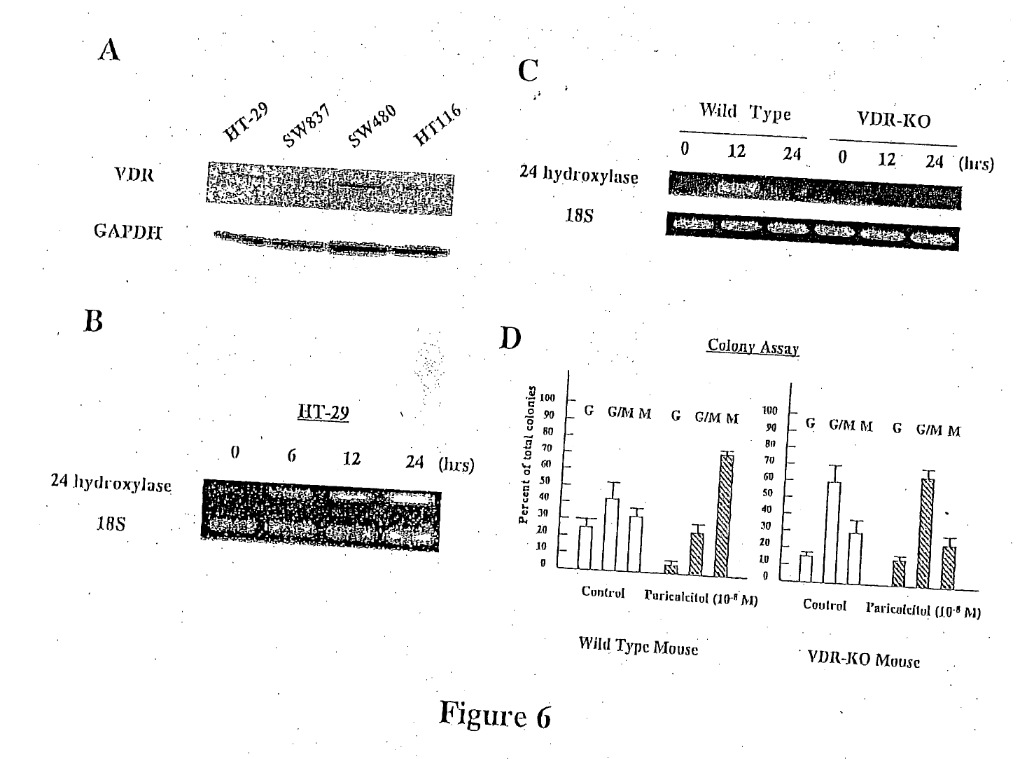

As disclosed herein, the role of the vitamin D receptor (VDR) in mediation of the effects of paricalcitol was evaluated. For example, each of the colon cancer cell lines used in FIG. 4 (HT-29, SW837, SW480, HCT116) was assayed for expression of the VDR protein. As shown in FIG. 6A, each of these cell lines expressed the VDR protein. When compared with the MTT antiproliferative results shown in FIG. 4A, no correlation was noted between the amount of VDR expressed by the various cell lines and their sensitivity to paricalcitol.

The enzyme responsible for the first step in the catabolism of 1,25(OH)₂D₃ is 25-hydroxyvitamin D3-24-hydroxylase (24-hydroxylase) (Jones et al., Physiol. Rev. 78:1193-1231 (1998)). Transcriptional induction of 24-hydroxylase is dependent on ligand activation of VDR and binding of the complex to the vitamin D response element of the promoter of the 24-hydroxylase gene (Zierold et al., Proc. Natl. Acad. Sci. USA 91:900-902 (1994); and Ohyama et al., J. Biol. Chem. 269:10545-10550 (1994)). As shown in FIG. 6B, paricarcitol (10⁻⁷M) induced expression of 24-hydroxylase mRNA within 6 hrs of exposure to HT-29 cells. In further experiments disclosed herein, mononuclear cells from spleens of either wild-type (WT) or VDR knock-out (KO) mice were isolated and cultured with paricarcitol (10⁻⁸ M). As shown in FIG. 6C, within 12 hours paricalcitol induced expression of 24-hydroxylase mRNA in the cells from the wild type mice, but not the VDR KO mice.

In further experiments disclosed herein, paricalcitol (10⁻⁸ M) was added to soft gel cultures of murine bone marrow cells from VDR knock-out and wild-type mice. After 2 weeks of culture, the colonies were scored as either macrophage, granulocyte or mixed granulocyte/macrophage colonies (O'Kelly et al., Clin. Invest. 109:1091-1099 (2002)). The number of total colonies (average) were 87 (control) and 66 (paricalcitol, 10⁻⁸ M) in wild type mice, and 110 (control) and 122 (paricalcitol, 10⁻⁸ M) in VDR knock out mice. As shown in FIG. 6D, addition of paricalcitol to soft-gel cultures altered the differentiation of committed myeloid stem cells from the wild-type mice, but not from the VDR knock-out mice. Examining myeloid stem cells in the wild-type mice, paricalcitol significantly increased the percentage of macrophage colonies (control, 32±4%; paricalcitol, 69±2%) and decreased the percentage of mixed colonies (control, 43±7%; paricalcitol, 25±5%) and granulocyte colonies (control, 23±4%; paricalcitol, 6±3%) (see FIG. 6D). Studying myeloid stem cells in the knock-out mice, paricalcitol did not alter the percentage of either macrophage (control, 30±8%; paricalcitol, 25±5%), mixed (control, 56±10%; paricalcitol, 61±8%) or granulocyte (control, 13±3%; paricalcitol, 13±4%) colonies (see FIG. 6D).

As disclosed herein, the vitamin D2 analog, paricalcitol, was able to inhibit the clonal proliferation of myeloid leukemia, myeloma, and colon cancer cell lines in vitro by modulating their cell cycle, differentiation and apoptosis. Furthermore, the compound was able to inhibit the in vivo growth of HT-29 human colon cancer tumors growing in nude mice.

The antiproliferative effects of paricalcitol on the tumor cells was associated with cell cycle arrest with corresponding changes of expression of CDKIs. Previous studies showed that vitamin D3 analogs can cause a G1/G0 cell cycle arrest, and this can be mediated by p21WAF1 and p27KIP1 (Wang et al., Cancer Res. 56:264-267 (1996); Munker et al., Blood 88:2201-2209 (1996) and Jiang et al., supra, 1994). Paricalcitol produced a G1/G0 and G2/M cell cycle arrest of HL-60 leukemia cells and a G1/G0 arrest of NCI-H929 myeloma cells, and induced expression of p21WAF1 and p27KIP1 in the leukemia, myeloma, and colon cancer cell lines. These results indicate that CDKIs can play a role in the antiproliferative effects of paricalcitol and other vitamin analogues by reducing the ability of the tumor cells to enter S phase (Doglioni et al., J. Pathol. 179:248-253 (1996) and Yang et al., Nat. Med. 1:1052-1056 (1995)). The block in the G2/M checkpoint has also been previously reported in HL-60 cells after their exposure to 1,25(OH)₂D₃ (Jiang et al., Oncogene 9:3397-3406 (1994)) similar to what is disclosed herein with paricalcitol. In a prior report, retardation of G2/M mediated by vitamin D3 was associated with decreased levels of p34(cdc) (Harrison et al., J. Cell. Biochem. 75:226-234 (1999)).

As disclosed herein, paricalcitol induced the expression of several tumor suppressor genes including PTEN and E-cadherin. The phosphatase PTEN can block the phosphatidylinositol 3′-kinase (PI3K)/Akt signaling pathways which can contribute to both cell death and inhibition of proliferation (Cantley and Neel, Prox. Natl. Acad. Sci. USA 96:4240-4245 (1999) and Di Cristof ano and Pandolfi, Cell 100:387-390 (2000)). Mutations of PTEN gene have been found in a variety of human cancers (Li et al., Science 275:1943-1947 (1997); Teng et al., Cancer Res. 57:5221-5225 (1997); Obata et al., Blood 58:2095-2097 (1998); Saki et al., Blood 92:3410-3415 (1998); Vlietstra et al., Cancer Res. 58:2720-2723 (1998); Dahia et al. Hum. Mol. Genet. 8:185-193 (1999); and Liu et al., Am. J. Hematol. 63:170-175 (2000)). While germline deletion of PTEN in the mice resulted in early embryonic lethality, heterozygous germline deletion of the gene was associated with an increased incidence of malignant neoplasms. These data indicate that PTEN behaves like a tumor suppressor gene depressing the pro-growth signals of the PI3 kinase pathway (Di Cristof ano et al., Nat. Genet. 19:348-355 (1998) and Suzuki et al., Curr. Biol. 8:1169-1178 (1998)). Previously it has been noted that 1,25(OH)₂D₃ and one of its analogs [21-(3-methyl-3-hydroxy-butyl)-19-nor D3] enhanced the levels of expression of PTEN in HL-60 cells (Histake et al., Blood 97:2427-2433 (2001)). This study showed that paricalcitol and 1,25(OH)₂D₃ induced PTEN expression in both HL-60 and NB-4 myeloid leukemia cells.

Paricalcitol or other vitamin D compounds can inhibit growth of cells having deletions of PTEN. Previously, an intragenic deletion including MMACl/PTEN exons 2-5 in the myeloblastic leukemia cell line HL-60, and an insertion of four nucleotides in exon 5 in an acute monocytic leukaemia cell line U937 were identified (Aggerholm et al. Eur. J. Haematol. 65:109-113 (2000)). These cells are induced to undergo terminal differentiation by vitamin D compounds. Furthermore, mutations of PTEN are present in prostate cancer cell lines (Li, supra, 1997). 1,25(OH)₂D₃ can inhibit the growth and induce the differentiation of these prostate cancer cells (Skowronski et al., Endocrinology 136:20-26 (1995); Danielpour et al., Cancer Res. 54:3413-3421 (1994); and Campbell et al., J. Cell. Biochem. 66:413-425 (1997)). Also, studies have shown that some tumor cells have low expression of PTEN associated with methylation of the promotor region of this gene including endometrial, breast, colon, and prostate cancer cell lines (Li et al., supra, 1997; Gunti et al., Human Mol. Genetics 9:283-287 (2000); and Tashiro et al. Cancer Res. 57:3935-3940 (1997)). Several vitamin D analogs have been shown to slow the growth of these cells. The antiproliferative activity of paricalcitol in these cells may be associated with their increased expression of PTEN and concomitant demethylation of the PTEN gene.

Concerning the NCI-H929 myeloma cells, as disclosed herein, induction of apoptosis by paricalcitol was accompanied by down-regulation of Bcl-2 protein expression without a change in levels of Bax protein. A previous study found that the vitamin D3 analogue, EB1089 was able to inhibit proliferation of NCI-H929 associated with apoptosis, downregulation of Bcl-2 expression and increased activity of caspase 3 (Park et al., Br. J. Heamatology 109:576-583 (2000)). Furthermore, EB1089 activated p38 kinase and suppressed p44 extracellular signal related kinase (ERK) activity during apoptosis of these cells.

Epidemiological studies suggested that the use of NTHEs decreased the risk of developing malignancies including colon cancer (Fosslien, Crit. Rev. Clin. Lab. Sci. 37:431-502 (2000) and Gupta, Nat. Rev. Cancer 1:11-21 (2001)). The major target of NTHEs is the COX family of enzymes that catalyze the conversion of arachidonic acid to prostaglandins (Seibert et al., Adv. Exp. Med. Biol. 400A:167-170 (1997)). COX-1 isozyme is expressed fairly ubiquitously in the body and is responsible for many physiological activities including the maintenance of the gastrointestinal mucosa as well as renal and platelet function (Siebert et al., Adv. Prostaglandin Thromoboxane Leukot. Res. 23:125-127 (1995) and Smith et al., Proc. Natl. Acad. Sci. USA 95:13313-13318 (1998)). In contrast, COX-2 is inducible by a variety of inflammatory stimuli, including cytokines, growth factors, and carcinogens; and it has been associated with promoting growth of cancerous and precancerous cells (Williams et al., Oncogene 18:7908-7916 (1999)). COX-2 expression is elevated in a variety of malignancies (va Rees and Ristimaki, supra, 2001; and Ristimaki et al., supra, 2002), and is, therefore, a reasonable target for chemoprevention of cancers. Selective COX-2 inhibitors suppress carcinogenesis in rodent models, and germline disruption of COX-2 inhibited polyp formation in APCD716-knockout mice (Oshima et al., Cell 87:803-809 (1996)). Furthermore, a selective COX-2 inhibitor reduced the polyp burden in patients with familial adenomatous polyposis (Steinbach et al., New England J. Medicine 342:1946-1952 (2000)). The Min mice (APC−/−) treated with vitamin D3 and its analog decreased total tumor load over the entire gastrointestinal tract (Huerta et al., supra, 2002).

As disclosed herein, paricalcitol and 1,25(OH)₂D₃ suppressed COX-2 but not COX-1 expression in the HT-29 and SW837 colon cancer cells indicating that this vitamin D analog acted as a selective COX-2 inhibitor. Some of anti-proliferative activity of vitamin D compounds against colon cancer cells may be associated with their inhibition of COX-2 expression.

Several experiments have been disclosed herein addressing whether paricalcitol mediated its effects through the vitamin D receptor (VDR). First, no correlation between levels of expression of VDR in colon cancer cell lines and their sensitivity to paricalcitol was found (FIG. 6A). It has previously been found that little correlation exists between overall cellular levels of VDR and responsiveness to vitamin D3 analogs (Koeffler et al., Mol. Cell. Endocrinology 70:1-11 (1990)). Some actions of vitamin D3 compounds can be mediated independently of the VDR by acting in a non-genomic pathway (Norman et al., J. Steroid Biochem. Mol. Biol. 41:231-240 (1992)). Nevertheless, as disclosed herein, paricalcitol induced 25-hydroxyvitamin D3-24-hydroxylase, a target gene of activated VDR. In addition, it was shown herein that this analog required the VDR to mediate macrophage differentiation of myeloid hematopoietic stem cells by comparing the ability of these committed myeloid stem cells to differentiate terminally when derived from VDR+/+ mice compared to VDR−/− mice. Also, 25-hydroxyvitamin D3-24-hydroxylase, a target gene of activated VDR, is not inducible by paricalcitol in the cells from VDR−/− mice, but is induced by paricalcitol in the VDR+/+ cells. Therefore, VDR can be necessary, but not sufficient to ensure that paricalcitol will have an antiproliferative effect on cancer cells.

As disclosed herein paricalcitol and 1,25(OH)₂D₃ had fairly comparable biological activities in vitro at similar concentrations. However, experience in humans has shown that paricalcitol is less likely to cause hypercalcemia, thus allowing the administration of higher doses and achieving higher peak serum concentrations of the analog. A typical dose of 1,25(OH)₂D₃ is 0.5 to 1.0 mg/every other day (QOD) achieving peak serum levels of 40-60 pg/ml. A typical dose of paricalcitol is 2.8-7.5 mg QOD and up to 16.8 mg has been safely given achieving peak serum levels of 1850 pg/ml. Furthermore, the serum half-life of both is similar. Therefore, paricalcitol can be given at higher doses, obtaining greater serum levels without toxicity compared to 1,25(OH)₂D₃.

Paricalcitol when combined with arsenic trioxide showed a markedly enhanced anti-proliferative effect against the myeloid leukemia cell lines, HL-60 and NB-4 as measured by MTT and colony assays compared to either drug alone. Paricalcitol (0.01 μM) alone induced monocytic differentiation of HL-60, while arsenic trioxide (0.8 μM) had little effect on differentiation, and when combined, the two drugs markedly enhanced monocytic differentiation of HL-60 as shown by NBT assay and induction of CD14 expression. The drug combination accumulated more HL-60 cells in G0/G1 cell cycle arrest and down-regulated Bcl-2 and Bcl-XL compared with treatment with either drug alone. Remarkably, neither paricalcitol (0.1 μM) nor arsenic trioxide (0.6 μM) induced differentiation of NB-4 APL cells, but the combination caused monocytic differentiation and subsequently marked apoptosis. To examine the association between the existence of the APL fusion protein and the effect of the combination treatment, U937 cells stably transfected with PML-RARA (PR9 cells) were cultured with paricalcitol. Paricalcitol induced monocytic differentiation in wild type U937 and vector-transfected U937, but differentiation was partially blocked in PR9. Arsenic trioxide, in a dose-dependent manner, decreased the levels of the original fusion protein in PR9, and the combination of paricalcitol and arsenic trioxide enhanced the differentiation of PR9 in parallel with an arsenic trioxide-induced decrease of PML-RARα, suggesting that the degradation of the fusion protein in promyelocytic leukemia cells by arsenic trioxide enhanced the ability of the combined therapy to induce differentiation of APL cells. Furthermore, arsenic trioxide decreased activity of the mitchondrial enzyme 24-hydroxyase (CYP-24), resulting in higher levels of the active vitamin D3 metabolite in HL-60 and NB-4 cells. In summary, paticalcitol and arsenic trioxide potently decreased growth and induced differentiation of APL cells, and this probably occurred by arsenic trioxide decreasing the PML-RARα fusion protein and CYP24 resulting in increased activity of the paricalcitol. The combination of both of these FDA-approved drugs should be considered for ATRA resistant APL patients.

The cell adhesion protein E-cadherin, which is also associated with differentiation, increased about 6-fold in the paricalcitol-treated and 1,25(OH)₂D₃-treated cultures (see FIG. 4B). E-cadherin is a transmembrane linker protein of the intercellular adherent junctions which maintains the adhesive and polarized phenotype of epithelial cells (Takeichi, Curr. Opin. Cell Biol. 7:619-627 (1995) and Gumbiner, Cell 84:345-357 (1996)). Loss of E-cadherin expression occurs during the transition from adenoma to carcinoma with the acquisition of capacity to invade (Perl et al., Nature 392:190-193 (1998) and Christofori and Semb, Trends Biochem. Sci. 24:73-76 (1999)). E-cadherin has been regarded as a tumor suppressor gene and its loss is often a predictor of poor prognosis. E-cadherin is also known as a regulator of b-catenin, holding it in place at the cell membrane. The loss of E-cadherin allows b-catenin to interact with cytoplasmic APC which helps mediate the ubiquitination and degradation of b-catenin. Mutation of the APC gene, which frequently occurs in the development of colon cancer, can result in b-catenin accumulating in the nucleus and acting as a co-stimulatory protein for the TCF family of transcription factors. Activation of these transcriptional factors stimulates a number of progrowth genes including cyclin D1 and c-myc (Polakis, Genes Dev. 14:1837-1851 (2000) and He et al., Science 281:1509-1512 (1998)).

A recent study suggested that ligand-activated VDR competed with TCF-4 for binding to b-catenin causing b-catenin to translocate from the nucleus back to the E-cadherin complex at the plasma membrane blunting the transcriptional regulatory activity of TCF (Palmer et al., J. Cell. Biol. 154:369-387 (2001)). As disclosed herein, paricalcitol increased the levels of E-cadherin and decreased expression of cyclin D1 and c-myc, the latter two being targets of TCF/b-catenin activation in HT-29 cells cultured with paricalcitol. Results disclosed herein are consistent with the anti-cancer effects of paricalcitol being associated with the modulation of the E-cadherin/b-catenin/TCF pathway. Of interest, it has been shown that Min mice (APC−/−) treated with 1,25(OH)₂D₃ or its analog had a decreased total tumor load over the entire gastrointestinal tract compared to control mice (Huerta et al., Cancer Res. 62:741-746 (2002)).

The COX enzymes catalyze the conversion of arachidonic acid to prostaglandins. Recently, elevated COX-2 expression has been associated with a variety of malignancies including colon cancer (van Rees and Ristimaki, Scand. J. Gastroenterol. 36:897-903 (2001) and Ristimaki et al., Cancer Res. 62:632-635 (2002)), and it has became a target for chemoprevention of several cancers including those of the colon. As shown in FIG. 4C, paricalcitol (10⁻⁷M, for 72 hrs) decreased the expression of COX-2 by 40% without affecting the expression of COX-1 in the HT-29 and SW837 colon cancer cells compared with diluant treated control cells. Under the same conditions, 1,25(OH)₂D₃ decreased levels of COX-2 by 50% (see FIG. 4C)

It is understood that modifications that do not substantially affect the activity of the various embodiments of this invention are also included within the definition of the invention provided herein. Accordingly, the following examples are intended to illustrate but not limit the present invention.

### EXAMPLE I

### Inhibition of Colony Formation by Paricalcitol

This example shows the inhibition of soft agar colony formation in myeloid leukemia, colon cancer, and myeloma cell lines by paricalcitol. The results are shown in FIG. 1.

A soft agar colony assay was used to test the effect of paricalcitol and 1,25(OH)₂D₃ on various cancer cell lines. For the soft agar colony assay, trypsinized and washed single-cell suspensions of cells were enumerated and plated into 24 well flat bottom plates using a two-layer soft agar system with a total of 1×10³ cells/well in a volume of 400 ml/well, as described previously (Kubota et al., supra, 1998).

Cell lines used in this study were obtained from American Type Culture Collection (Rockville, Md.) and were maintained according to their recommendations. Myeloid leukemia cell lines (HL-60, NB-4, THP-1, U937), lymphoma cell lines (Raji, Ramos, Daudi, Jurkat, Jeko-1, JUDHL) and myeloma cell lines (RPMI-8226, ARH-77, NCI-H929) were grown in RPMI 1640 with 10% FCS. Breast cancer cell lines (MCF-7, MDA-MB-231), brain cancer cell lines (U343, U118, U138, U373, U87), and colon cancer cell lines (HT-29, SW837, SW480, SW620, HCT116) were maintained in DMEM with 10% FCS. Endometrial carcinoma cell line, AN-3 was maintained in Alpha Minimum Essential Medium (a-MEM) with 10% FCS.

### EXAMPLE II

### Paricalcitol Effect on Cell Cycle and Differentiation

This example shows that paricalcitol affects cell cycle and differentiation status of myeloid leukemia cells.

For cell cycle analysis, cells were exposed to 10⁻⁷M 1,25(OH)₂D₃, 10⁻⁷M paricalcitol or vehicle control for either 3 or 4 days. Total cells, both in suspension and adherent, were collected, washed, suspended in cold PBS. Then cells were fixed in 75% chilled methanol and stained with propidium iodine. Cell cycle status was analyzed on a Becton Dickinson Flow Cytometer. The results are shown in FIG. 2A.

Western blot analysis was used to determine the levels of proteins involved in cell cycle and differentiation. For western blot analysis, cells were washed twice in PBS, suspended in lysis buffer [50 mM Tris (pH 8.0), 150 mM NaCl, 0.1% SDS, 0.5% sodium deoxycholate, 1% NP40, 100 mg/ml phenylmethylsulfonyl fluoride, 2 mg/ml aprotinin, 1 mg/ml pepstatin, and 10 mg/ml leupetin] and placed on ice for 30 min. After centrifugation at 15,000×g for 15 min at 4° C., the suspension was collected. Protein concentrations were quantitated using the Bio-Rad assay (Bio-Rad Labolatories, Hercules, Calif.). Whole lysates (40 mg) were resolved by 4-15% SDS polyacrylamide gel, transferred to an immobilon polyvinylidene difuride membrane (Amersham Corp., Arlington Heights, Ill.), and probed sequentially with a number of antibodies (p21WAF1, p27KIP1, PTEN, and GAPDH, Santa Cruz Biotechnology Inc., Santa Cruz, Calif.). The blots were developed using the Supersignal West Pico Chemiluminescent Substrate Kit (Pierce, Rockford, Ill.). The results are shown in FIG. 2B.

In order to measure cell surface CD14 antigen on HL-60 cells, promyelocytic leukemia cell line, HL-60, was treated with either 1,25(OH)₂D₃ or paricalcitol (10⁻⁷M) for 4 days and examined for CD14 expression by flow cytometry using CD14 antibody (DAKO, Carpinteria, Calif.), as described previously (Hisatake et al., supra, 2001). Murine IgG1 antibody (DAKO) was used as a control. The results are shown in FIG. 2C.

### EXAMPLE III

### Paricalcitol Effect on Cell Cycle and Apoptosis

This example shows that paricalcitol affects cell cycle and apoptosis status of NCI-H929 cells.

Cell cycle analysis of NCI-H929 cells by flow cytometry was performed and is shown in FIG. 3A. HCI-H929 cells were cultured with either paricalcitol (10⁻⁷M) or 1,25(OH)₂D₃ (10⁻⁷M) for 72 hrs, harvested and stained with propidium iodine (PI). Control cells were treated with vehicle alone.

In FIG. 3B, quantitive analysis of apoptosis of NCI-H929 cell line exposed to either paricalcitol (10⁻⁷M), or 1,25(OH)₂D₃ (10⁻⁷M) for 96 hrs and analyzed by TUNEL assay is shown. Results represent the mean±SD of three independent experiments. A TUNEL assay was performed for immunohistochemical detection and quantification of programmed cell death at the single cell level, based on labeling of DNA strand breaks using the In Situ Cell Death Detection, POD (Roche, Indianapolis, Ind.). Early apoptosis was also detected by measuring annexin V protein in the cell membrane using Annexin V-FITC Kit (CLONTECH, Palo Alto, Calif.) followed by flow cytometric analysis. The results are shown in FIG. 2B.

In FIG. 3C, NCI-H929 cells were treated with either paricalcitol (10⁻⁷M) or 1,25(OH)₂D₃ (10⁻⁷M) and cell lysates were prepared after 72 hrs. Cell lysates were used for Western blot analysis and probed sequentially with antibodies to p27KIP1, Bcl-2 and Bax. Control cells were treated with vehicle alone. Amount of protein was normalized by comparison to the amount of GAPDH. Western blot analysis of NCI-H929 cells treated with paricalcitol or 1,25(OH)₂D₃ was performed as described for Example II. The Bcl-2 and Bax antibodies were obtained from Santa Cruz Biotechnology Inc., Santa Cruz, Calif. The results are shown in FIG. 3C.

### EXAMPLE IV

### Effect of Paricalcitol on Colon Cancer Cells

This example shows the effect of paricalcitol on colon cancer cell lines HT-29, SW837, SE480 and HT116.

HT-29, SW837, SW480 and HCT116 colon cancer cells were treated for 96 hrs with either paricalcitol (10⁻⁷M) 1,25(OH)₂D₃ (10⁻⁷M) or diluant (control). Growth (% of control) was measured by MTT assay. Results represent the mean±SD of three independent experiments with triplicate dishes. For the MTT assay, MTT (Sigma) was placed in solution with PBS (5 mg/ml) and used to measure either cellular proliferation or viability. 103 cells were incubated in culture medium for 96 hr in 96 well-plates and 10 ml of MTT solution was added. After 4 hrs incubation, 100 ml of solubilization solution (20% SDS) was added, and the solution was incubated at 37° C. for 16 hr. In this assay, MTT is cleaved to an orange formazan dye by metabolically active cells. The dye was directly quantified using an enzyme-linked immunoabsorbent assay reader at 540 nm.

In FIG. 4B, HT-29 cells were exposed to either paricalcitol (10⁻⁷M) or 1,25(OH)₂D₃(10⁻⁷M). Cell lysates were prepared after 72 hrs of culture and analyzed by Western blot. The Western blot was probed sequentially with antibodies for p27KIP1, p21WAF1, cyclin D1, c-myc and E-Cadherin (antibodies from Santa Cruz Biotechnology) as described in Example II. Control cells were treated with vehicle alone. The quantity of protein was normalized by comparison to the amount of GAPDH.

In FIG. 4C, HT-29 and SW837 cells were cultured with either paricalcitol (10⁻⁷M) or 1,25(OH)₂D₃ (10⁻⁷M) for 72 hrs. Cell lysates were prepared and analyzed by Western blot which was probed sequentially with antibodies to COX-1 and COX-2 (Santa Cruz Biotechnology). Control cells were treated with vehicle alone. The amount of protein was normalized by comparison to the quantity of GAPDH.

### EXAMPLE V

### Effect of Paricalcitol on Colon Cancer Cells In Vivo

FIG. 5 shows effects of paricalcitol on the growth of HT-29 colon cancer cells growing as tumors in nude mice. HT-29 cells were bilaterally injected subcutaneously into nude mice, forming two tumors per mouse. The mice were divided randomly into control and experimental groups. Paricalcitol (100 ng/mouse) was administered intraperitoneously for 3 days a week in the experimental groups (Monday, Wednesday, Friday).

In FIG. 5A, tumor volumes were measured every week. The mean volume±SD of 10 tumors in each group is shown. Tumor volumes were significantly different between the experimental and control groups (p=0.03). In FIG. 5B, After 4 weeks of therapy, tumors were removed from each group and weighed. The tumor weights were significantly different in the two groups (p=0.0004).

For the mouse studies, BNX nu/nu nude mice at 8 weeks of age were purchased from Harlan Sprangue Dawley, Inc. (Indianapolis, Ind.) and their care was in accord with the guidelines of Cedars-Sinai Research Institute. They were maintained in pathogen-free conditions with irradiated chow. A total of 1×10⁶ HT-29 cells in 0.1 ml of Matrigel (Collaborative Biological Products, Bedford, Mass.) were injected s.c. into bilateral flanks of each mouse, resulting in the formation of two tumors per mouse. The mice were blindly randomized to the experimental and control groups. Treatment was started on the day after the injection of PC-3 cells and continued for 6 weeks. The control mice (five) received diluant only and the experimental mice (five) received paricalcitol [100 ng/day, intraperitoneally, 3 days per week (M,W,F)]. Tumor sizes were measured every week and calculated by the formula: A (length)×B (width)×C (height)×0.5236. After 4 weeks, blood was collected for serum calcium. All mice were euthanized at the end of the study, and the tumors were fixed in 10% neutral buffered formalin and embedded in paraffin for histological analysis. The data were analyzed by Student's t test.

To measure the serum calcium levels in mice, Sigma Diagnostics calcium reagent (Sigma, MO) containing o-cresolphthalein, which complexes with calcium to form a purple colored complex, was used. The colored complex was directly quantified using an enzyme-linked immunoabsorbent assay reader at 575 nm.

### EXAMPLE VI

### The Role of Vitamin D Receptor (VDR) in Paricalcitol Action

FIG. 6 shows expression of vitamin D receptor (VDR) in cell lines, expression of 24-hydroxylase in response to paricalcitol, and the effect of paricalcitol in cells isolated from wild-type and VDR knock out mice. In FIG. 6A, cell lysates of HT-29, SW837, SW480, SW620 and HCT116 colon cancer cells were harvested and VDR expression was measured by Western blot. The amount of protein was normalized by comparison to levels of GAPDH. Western blots were performed as described above,

In FIG. 6B, HT-29 colon cancer cells were treated with paricalcitol (10⁻⁷M) for 0, 6, 12 or 24 hrs and RNA was harvested. Expression of 24 hydroxylase mRNA was analysed by RT-PCR. The amounts of mRNA were normalized by comparison to 18S RNA. In FIG. 6C, mononuclear cells extracted from spleens of either wild type or VDR knock-out mice were treated with paricalcitol (10⁻⁸M) for either 12 or 24 hrs, and RNA was harvested. Expression of 24 hydroxylase mRNA was analysed by RT-PCR. The amounts of mRNA were normalized by comparison to 18S RNA. For the PCR analysis, RNA extraction and reverse transcription were done by TRIzole (Invitrogen, Carlsbad, Calif.) and reverse transcriptase (Promega, Madison, Wis.). A twenty-microliter volume of cDNA was prepared from 1 μg of RNA. cDNAs were amplified by PCR with specific primers for 24-hydroxylase and 18S. The cycle number was 25 for 18S and 32 for 24-hydroxylase. PCR product was separated on a 2% agarose gel, stained with ethidium bromide, and photographed.

In FIG. 6D, colony formation by mononuclear bone marrow cells from VDR knock-out (VDR-KO) and wild type (WT) mice is shown. Mononuclear cells were obtained from femoral bone marrow plugs and grown in methylcellulose culture media with either paricalcitol (10⁻⁸M) or diluant. Colonies were counted on day 10 of culture. The number of total colonies (average) were 87 (control) and 66 (paricalcitol 10⁻⁸M) in wild type mice, and 110 (control) and 122 (paricalcitol 10⁻⁸M) in VDR-KO mice. The percentage of macrophage, granulocyte and mixed granulocyte/macrophage colonies are shown. Triplicate wells for each mouse and a total of three KO and three WT mice were studied. G, granulocyte colonies; G/M mixed granulocyte/macrophage colonies; M, macrophage colonies.

The VDR KO Mouse and Colony-Forming Assay was performed as follows. VDR KO mice were generated and genotypes were determined by Southern Blot Analysis as described previously (O'Kelly et al., supra, 2002). For experiments using VDR KO mice, their wild-type (WT) littermates were used as controls. Mice were killed by cervical neck dislocation. Bone marrow was flushed out of isolated femurs with (a-MEM; Gibco BRL, Grand Island, N.Y., USA) including 10% FCS using a 26-gauge needle. Isolated spleens were injected with DMEM (Gibco BRL) plus 10% FCS and crushed with forceps to release cells. Mononuclear cells from bone marrow or spleen were separated by Ficoll-Hypaque density centrifugation (Amersham Pharmacia, Uppsala, Sweden). Resuspended mononuclear bone marrow cells (2×10⁴ cells/ml) and growth factors were added 1:10 to methylcellulose medium M3234 (StemCell Technologies Inc., Vancouver, British Columbia, Canada) to yield a final concentration of 1% methylcellulose, 30% FCS, 1% BSA, 10-4M mercaptoethanol, and 2 mM L-glutamine as described previously (19). Cells were plated in six-well plates in a volume of 1 ml and incubated at 37° C. in a humidified atmosphere containing 5% CO2. Colonies were counted after 2 weeks. Colony type was established by morphology; and to ensure accurate determination, representative colonies were plucked from the plates, centrifuged onto slides, stained with Wright-Giemsa stain and examined by light microscopy.

### EXAMPLE VII

### Combination of Paricalcitol and Arsenic Trioxide Inhibits Cell Proliferation

This example shows that paricalcitol in combination with arsenic trioxide had prominent antiproliferative activity against human myeloid leukemia cells.

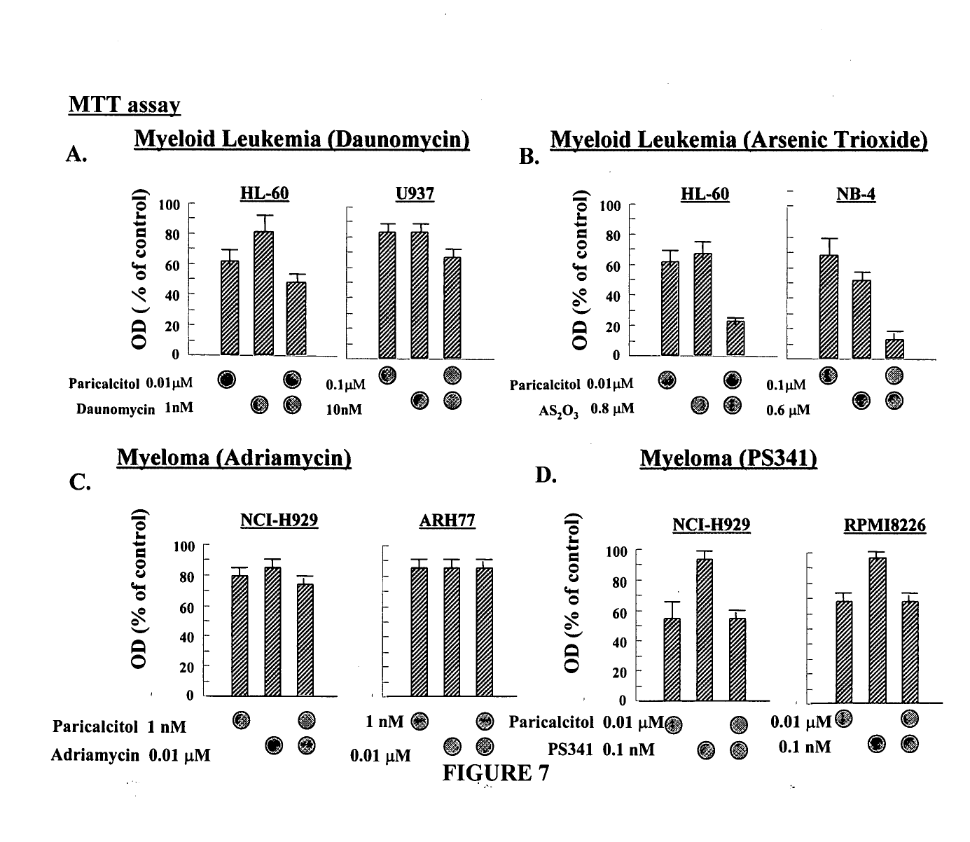

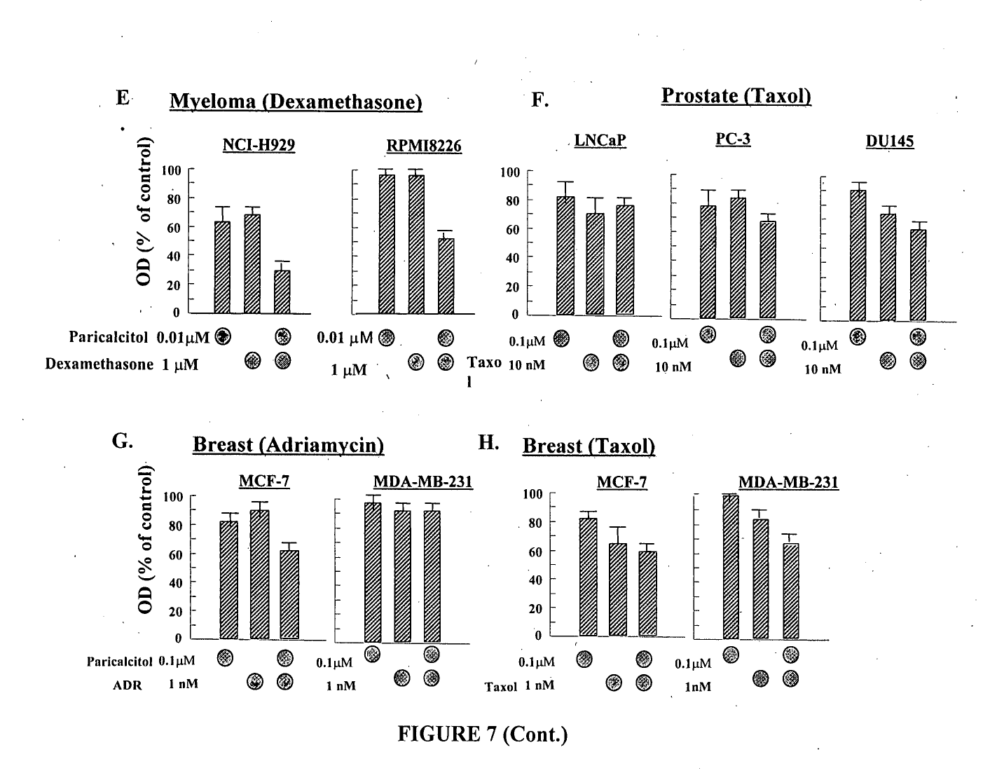

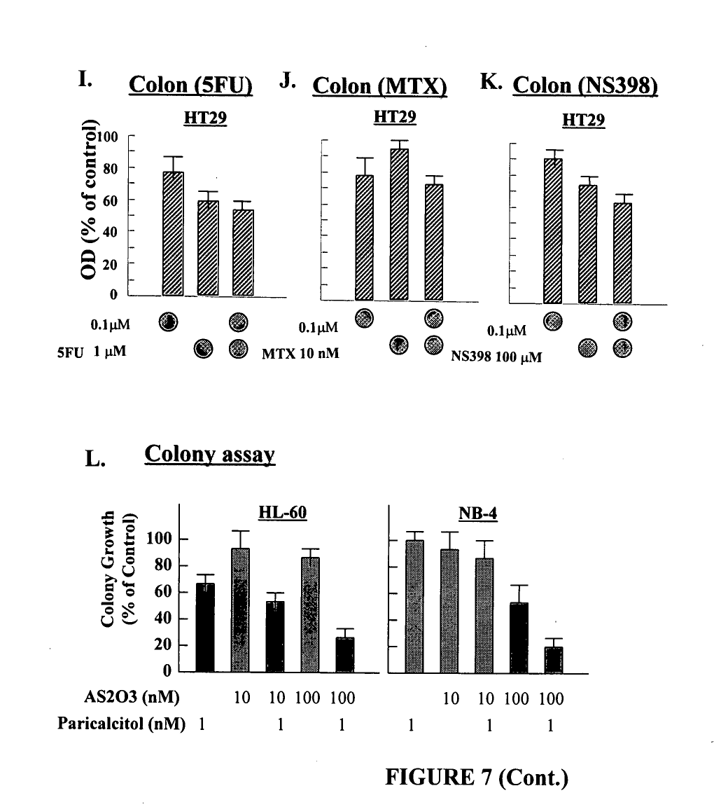

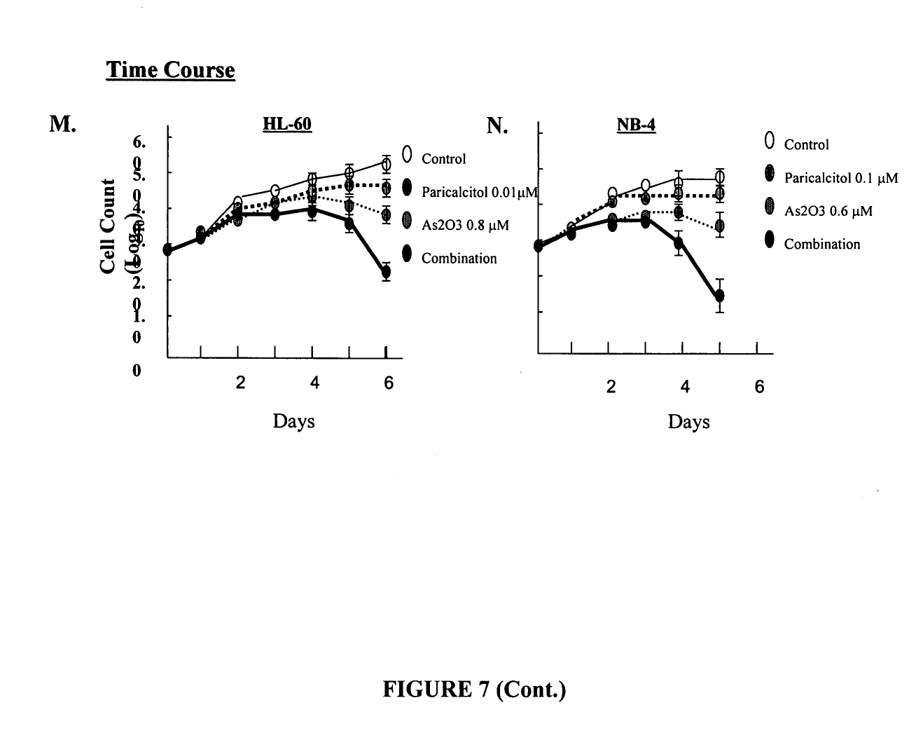

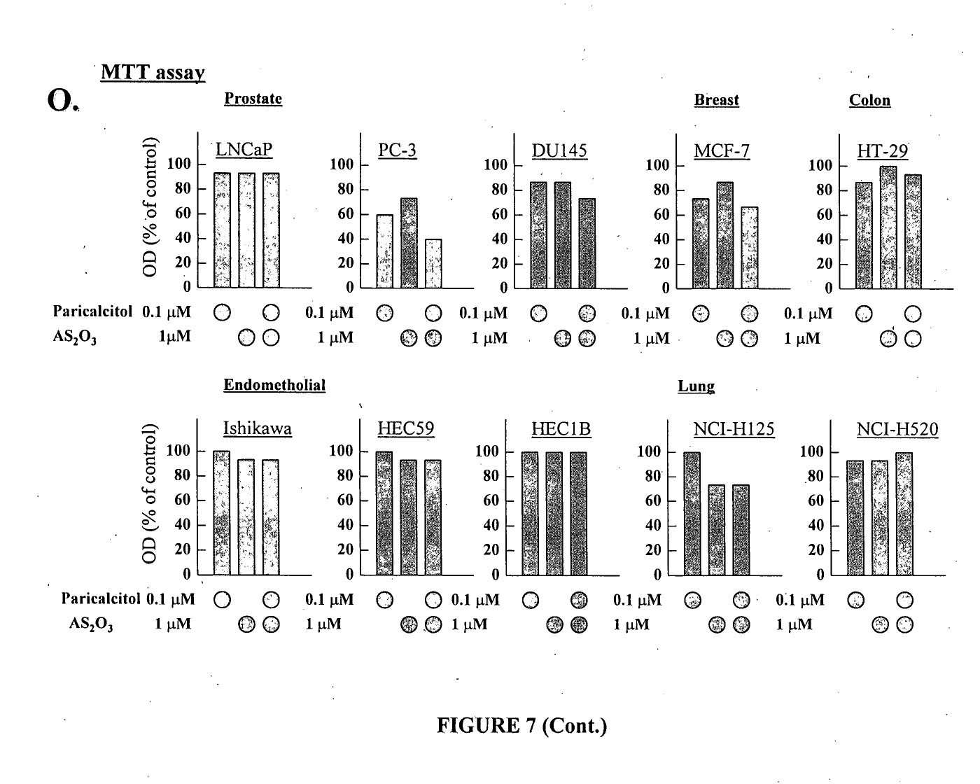

The anti-proliferation effect of vitamin D2 analog, paricalcitol in combination with other clinically-used anti-cancer agents was examined on various cancer cell lines in vitro. Paricalcitol was used in combination with daunorubicin and arsenic trioxide to treat myeloid leukemia cells (HL-60, NB-4, U937); in combination with doxorubicin (adriamycin), vincristine or dexamethasone to treat multiple myeloma cells (NCI-H929, RPMI8226, ARH-77); in combination with taxol to treat prostate cancer cells (LNCaP, PC-3, DU145); in combination with doxorubicin or taxol to treat breast cancer cells (MCF7, MDA-MB-231); and in combination with doxorubicin, 5-FU or COX-2 inhibitor (NS-384) to treat colon cancer cell (HT-29). Cancer cell lines were treated with these combinations, with the first screening performed using the rapid MTT assay with a relative short exposure of 4 days to the agents (FIGS. 7A-K). Various concentrations of each drug was used, and the results were shown in the case of the indicated concentration. Among these combinations, it was found that the combination of paricalcitol and arsenic trioxide had prominent antiproliferative effect against myeloid leukemia cells, (HL-60, NB-4) compared to each drug alone. Each drug suppressed the cell growth in a dose-dependent manner in both cell lines, and these two drugs had synergestic effects on these cells. The combination of paricalcitol and dexamethasone also had significant anti-proliferative effect on multiple myeloma cells (FIG. 7B). These two drugs had also synergistic anti-proliferative effects on both cell lines. The combination treatment of paricalcitol and arsenic trioxide was used to treat myeloid leukemia cells. This combination suppressed colony growth of both cells by colony assay (FIG. 7L). The time course assay of cell numbers counted by trypan blue assay, when both cells were treated with paricalcitol (0.01 μM for HL-60, 0.1 μM for NB-4) and arsenic trioxide (0.8 μM for HL-60, 0.6 pM for NB-4) are also shown (FIGS. 7M and N). In the time course, dead cells were not prominent during the first few days by trypan blue assay, but after 3 or 4 days cells began to die, especially in NB-4 cells. Prostate (LNCap, PC-3, DU145), breast (MCF-7), colon (HT-29), endometholial (Ishikawa, HEC59, HEC1B) and lung (NCI-H125, NCI-H520) cancer cell lines were treated with paricalcitol (0.1 μm) and arsenic trioxide (1 μm), and the MTT assay was performed after 4 days. This combination also showed additive antiproliferative effects on PC-3 prostate cancer cells (FIG. 70).

Cell lines used in this study were obtained from American Type Culture Collection (Rockville, Md.) and were maintained according to their recommendations. Myeloid leukemia cell lines (HL-60, NB-4, THP-1, U937), lymphoma cell lines (Raji, Ramos, Daudi, Jurkat, Jeko-1, JUDHL), myeloma cell lines (RPMI-8226, ARH-77, NCI-H929), ovarian cancer cell lines and PC-3 prostate cell line were grown in RPMI 1640 with 10% FCS. Breast cancer cell lines (MCF-7, MDA-MB-231), colon cancer cell lines (HT-29, SW837), pancreatic cancer cell lines, and endomethorial cancer cell lines were maintained in DMEM with 10% FCS. Compounds other than paricalcitol and PD58048 were obtained from Sigma. For induction of PML-RAR in PR9 cells, 0.1 mmol/L ZnSO₄ was added to the culture media.

For MTT assays, MTT (Sigma) was placed in solution with PBS (5 mg/ml) and used to measure cellular proliferation. After 10³ cells were spread in 96 well-plates, they were incubated in culture medium containing some drug for 96 hours and 10 μl of MTT solution was added. After 4 hrs incubation 100 μl of solubilization solution (20% SDS) was added and incubated at 37° C. for 16 hours. In this assay, MTT is cleaved to an orange formazan dye by metabolically active cells. The dye was directly quantified using an enzyme-linked immunoabsorbent assay reader at 540 nm.

### EXAMPLE VIII

### Combination of Paricalcitol and Arsenic Trioxide Enhances Monocytic Differentiation and Increases Apoptosis

This example shows that paricalcitol combined with arsenic trioxide markedly enhanced monocytic differentiation of HL-60 and NB-4 myeloid leukemia cells with subsequently increasing apoptosis.

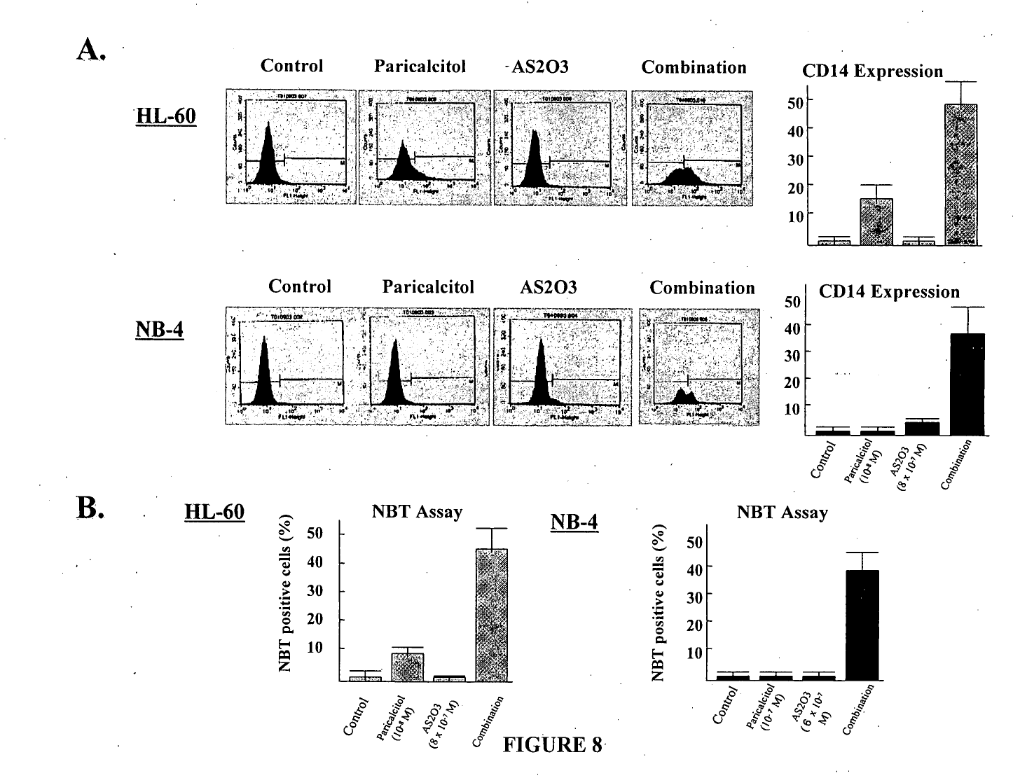

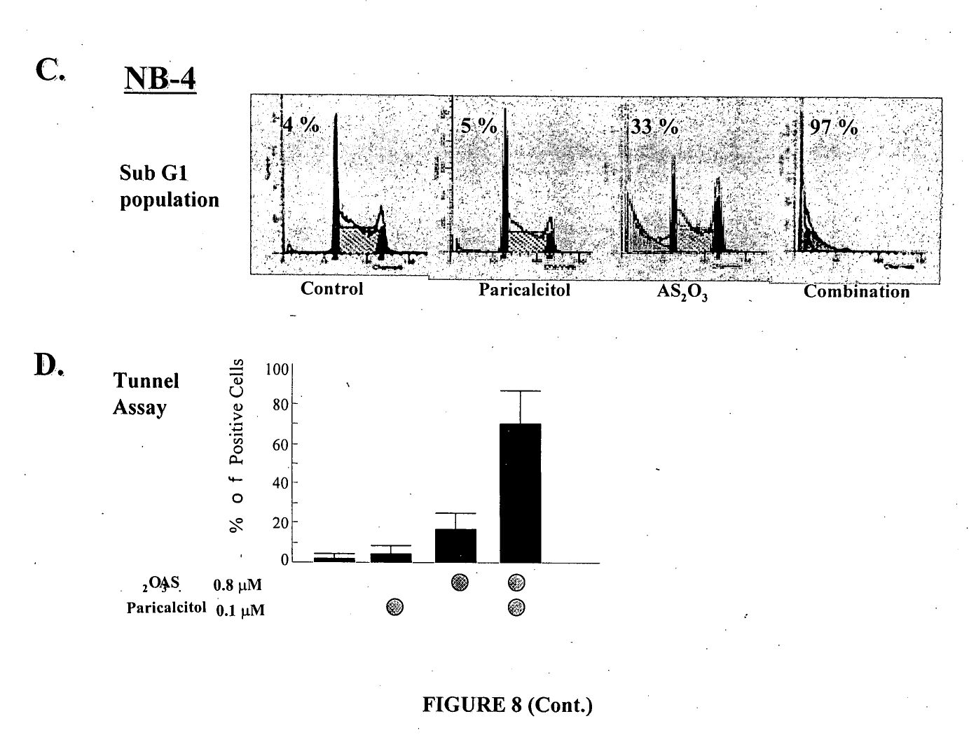

The effect of paricalcitol in combination with arsenic trioxide on differentiation and apoptosis of HL-60 and NB-4 was examined. After treatment with paricalcitol (0.01 μM for HL-60, 0.1 μM for NB-4) and arsenic trioxide (0.8 μM for HL-60, 0.6 μM for NB-4) for 3 days, CD14, a marker of monocytic differentiation was measured by flow cytometry in HL-60 and NB-4 cells. In HL-60 cells, paricalcitol alone increased cell surface CD14, but arsenic trioxide did not. The combination of these drugs markedly induced CD14 expression compared to paricalcitol alone (FIG. 8A). Neither paricalcitol nor arsenic trioxide alone did not induced CD14 expression in NB-4 cells, but it is notable that paricalcitol when combined with arsenic trioxide induced CD14 expression in this cell line (FIG. 8A). Monocytic differentiation was also measured by NBT reduction in HL-60 and NB-4 cells after treatment with paricalcitol and/or arsenic trioxide (FIG. 8B). In HL-60, NBT reduction was increased by paricalcitol, and arsenic trioxide enhanced the increase of paricalcitol. In NB-4 cells, paricalcitol, when only combined with arsenic trioxide, increased the NB-4 reduction.

After treatment of HL-60 and NB-4 cells with paricalcitol and arsenic trioxide, dead cells were detected by trypan blue assay after 5 to 6 days in HL-60, and after 3 to 4 days in NB-4 cells. Based on these observations, apoptotic cells were examined by measurement of sub-G1 population in cell cycle analysis and TUNEL assays. After treatment of NB-4 cells with paricalcitol (0.1 μM) and/or arsenic trioxide (0.6 μM) for 4 days, arsenic trioxide alone induced apoptosis as shown by cells in sub-G1 population (33%), while control cells and paricalcitol-treated cells showed 4% and 5% of cells in the sub-G1 population, respectively. The combination of two drugs increased apoptotic cell death with 97% of cells in sub-G1 population (FIG. 8C). Also determined was the percentage of apoptotic cells by TUNEL assay after treatment with paricalcitol (0.01 μM for HL-60, 0.1 μM for NB-4) and/or arsenic trioxide (0.8 μM for HL-60, 0.6 μM for NB-4). The combination increased apoptosis compared to each drug alone (FIG. 8D). In HL-60 cells, apoptosis was also detected by determining the percentage of cells in the sub-G1 population and by TUNEL assay after 6-7 days treatment with paricalcitol and arsenic trioxide.

Cell cycle analysis was performed as follows: after treatment of 5×10⁴ of cells with a selected compound, cells were collected, washed and suspended in cold PBS. Cells were fixed in chilled 75% methanol and stained with propidium iodine. Cell cycle status was analyzed on a Becton Dickinson Flow Cytometer used standart protocols.

Apoptosis was determined using the TUNEL assay for immunohistochemical detection and quantification of programmed cell death at the single cell level, based on labeling of DNA strand breaks using the In Situ Cell Death Detection, POD (Roche, Indianapolis, Ind.).

Measurement of cell surface CD14 antigen on HL-60 cells by flow cytometry using CD14 antibody (DAKO, Carpinteria, Calif.), was determined as described previously (Hisatake et al. Blood 97:2427-2433 (2001)). Murine IgG1 antibody (DAKO) was used as a control.

### EXAMPLE IX

### Combination of Paricalcitol and Arsenic Trioxide Alters Gene Expression in Myeloid Leukemia Cells

This example shows that expression of several genes is modulated by paricalcitol and arsenic trioxide in myeloid leukemia cells.

HL-60 and NB-4 cells were used to examine gene expression in response to treatment with paricalcitol and arsenic trioxide. The enzyme 25-hydroxyvitamin D3-24-hydroxylase catalyzes the first step in the catabolism of 1,25(OH)₂D₃. Expression of 24-hydroxylase is transcriptionally regulated and is activated by the binding of its ligand, 1,25(OH)₂D₃ or its analog to the VDR. The VDR-ligand complex (VDR-RXR) then binds to the vitamin D response element in the 24-hydroxylase promoter and activates its transcription. It was determined that the expression of VDR and RXR were not substantially changed by paricalcitol and/or arsenic trioxide in HL-60.

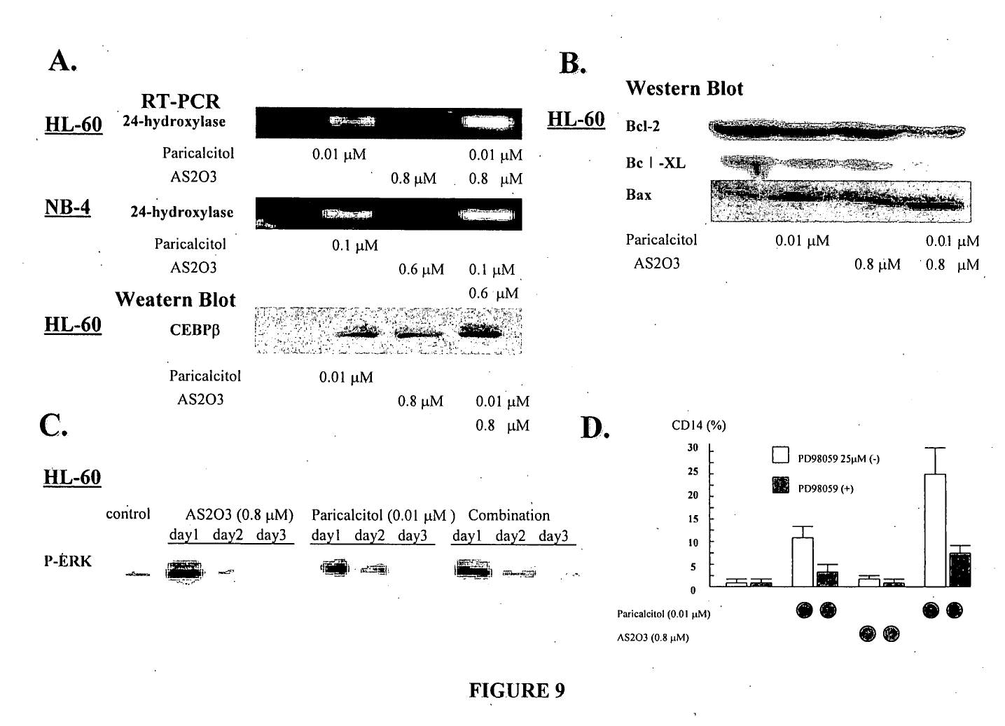

The expression of 24-hydroxylase as an early vitamin D target gene was examined by RT-PCR. The mRNA levels of 24-hydroxylase were increased after treatment of paricalcitol alone (0.01 μM for HL-60, 0.1 μM for NB-4) after 24 hrs (FIG. 9A, B). When the cells were treated with paricalcitol and arsenic trioxide (0.8 μM for HL-60, 0.6 μM for NB-4), the transcriptional levels of 24-hydroxylase were much higher than paricalcitol alone, indicating that arsenic trioxide enhanced the transcriptional activation through VDR (FIG. 9A, B).

The expression of C/EBPβ was examined by western blot analysis. In HL-60 cells, expression of C/EBPβ was increased by paricalcitol. Arsenic trioxide alone also increased its expression level, and the combination of both drugs increased its expression more than each drug alone.

There are several genes involved in the differentiation induced by vitamin D in myeloid cells. In HL-60 cells, phosphorylated Rb gene activated by 1,25(OH)D₃ may be associated with the quiescent state in cell cycle regulation during differentiation. Therefore, phosphorylation of Rb gene was examined after treatment with paricalcitol and/or arsenic trioxide for 3 days. It was shown that the phosphorylation of Rb increased upon treatment with paricalcitol and arsenic trioxide, and the combination increased its expression stronger.

After enhancement of differentiation by the combination of paricalcitol and arsenic trioxide, HL-60 and NB-4 underwent apoptosis (FIG. 9B). Levels of expression of antiapoptotic genes Bcl-2 and Bcl-XL significantly decreased after the treatment with paricalcitol (0.01 μM) and arsenic trioxide (0.8 μM) although expression of proapoptotic gene Bax was not changed by either drug or both in HL-60 cells.

Activated ERK has been reported to have an important role in monocytic differentiation induced by vitamin D3 in HL-60 cells. Therefore, the expression of phosphorylated-ERK was examined by western blot. It was observed that expression was increased after exposure of HL-60 cells to paricalcitol (0.01 μM) and was also increased after exposure to arsenic trioxide (0.8 μM) alone or in combination with paricalcitol. The expression levels were peak after exposure to each drug for 1 day (FIG. 9C). To determined if increased expression of p-ERK in response to both drugs is important for enhanced differentiation induced by the combination treatment, HL-60 cells were treated with a potent selective MAPK/ERK kinase inhibitor, PD98059. HL-60 cells were treated with PD98059 (25 μM) in combination with paricalcitol and/or arsenic trioxide, and CD14 expression (a marker of monocytic differentiation) was measured by flow cytometry (FIG. 9D). When HL-60 cells were treated with paricalcitol and/or arsenic trioxide in the presence of PD98059, differentiation induced by paricalcitol was decreased and was not significantly enhanced by the combination treatment. This indicated that monocytic differentiation induced by paricalcitol and the enhanced differentiation by the combination were blocked by PD98059. These results indicate that activation of ERK is necessary for enhancement of paricalcitol-induced differentiation by arsenic trioxide when HL-60 cells were treated with the both drugs.

Western blot analysis was performed as follows: cells were washed twice in PBS, suspended in lysis buffer (50 mM Tris at pH 8.0, 150 mM NaCl, 0.1% SDS, 0.5% sodium deoxycholate, 1% Nonidet P-40, phenylmethylsulfonyl fluoride at 100 μg/mL, aprotinin at 2 μg/mL, pepstatin at 1 μg/mL, and leupetin at 10 μg/mL), and placed on ice for 30 minutes. After centrifugation at 15,000×g for 15 minutes at 4° C., the suspension was collected. Protein concentrations were quantitated by using the Bio-Rad protein Assay Dye Reagent Concentrate (Bio-Rad Laboratories, Hercules, Calif.) according to the manufacture's recommendation. Whole cell lysates (40 μg) were resolved by SDS-polyacrylamide gel electrophoresis in a 4%-15% gel, transferred to a polyvinylidene difuride membrane (Immobilon, Amersham Corp., Arlington Heights, Ill.), and probed sequentially with antibodies against the following proteins: VDR, RXR, CEBPβ, Rb, Bcl-2, Bcl-XL, Bax, p-ERK, RARA, GAPDH (Santa Cruz Biotechnology Inc., Santa Cruz, CA). The blots were developed using the Supersignal West Pico Chemiluminescent Substrate Kit (Pierce, Rockford, Ill.).

### EXAMPLE X

### Combination of Paricalcitol and Arsenic Trioxide Overcomes Inhibition of Differentiation Induced by PML-RARA

This example shows that paricalcitol in combination with arsenic trioxide overcomes the block of differentiation induced by PML-RARA fusion protein.

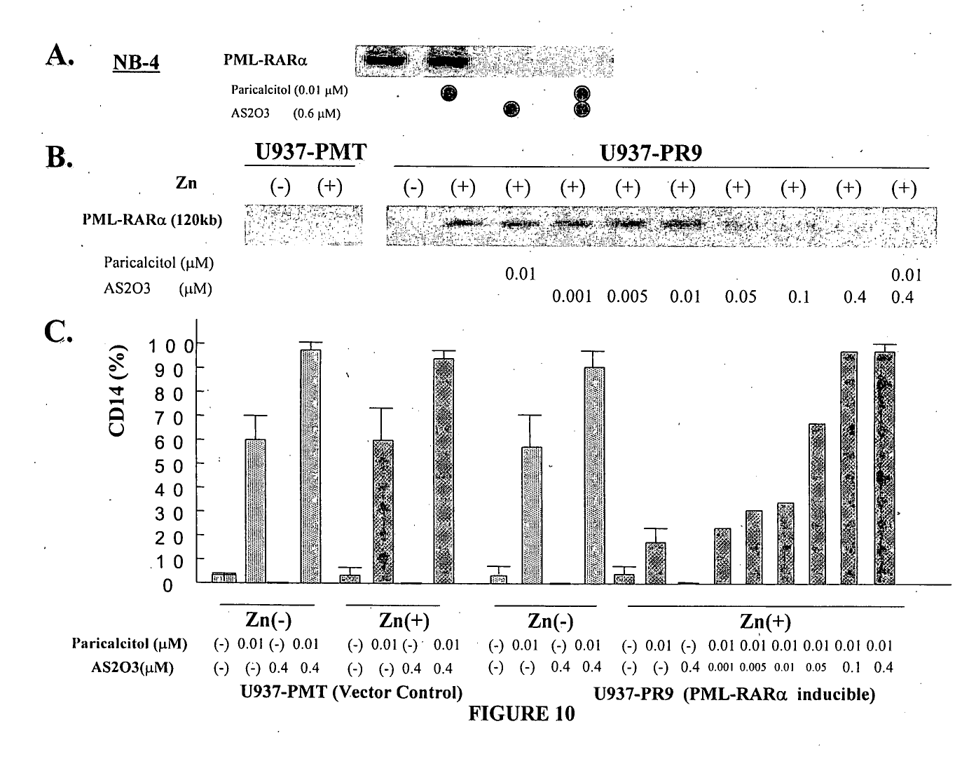

NB4 promyelocytic leukemia cells express PML-RAR fusion gene which has important role in the pathogenesis of acute lymphocytic leukemia. Arsenic trioxide which is used as a therapeutic agent for acute lymphocytic leukemia, is reported to degradate this fusion protein. As is shown below, arsenic trioxide decreased the protein level of PML-RAR after treatment with arsenic trioxide (0.6 μM) for 3 days and its expression could not be detected by western blot using anti-RARa antibody when NB-4 cells were treated with arsenic trioxide (0.6 μM) or arsenic trioxide with paricalcitol (0.1 μM) (FIG. 10A).

To examine the association between the presence of the fusion protein and the effect of the combination treatment, an engineered U937 monocytic leukemia cell line (PR9) that has stable integration of the PML-RARa cDNA under the control of the Zn²⁺-inducible murine metallothionein 1 promoter was used. Also used were U937 cells transfected with the MT vector (B41) as a control. Cell surface marker, CD14 was used as a marker for monocytic differentiation. When wild type U937 cells were cultured with paricalcitol (0.01 μM) and/or arsenic trioxide (0.4 μM), paricalcitol increased CD14 expressing monoicytic cells, and the combination enhanced the increase of CD14 expressing cells. Arsenic trioxide alone did not induce CD14 expression (FIG. 10C). This indicated that paricalcitol in combination with arsenic trioxide also enhanced paricalcitol-induced monocytic differentiation in U937 cells. The combination also enhanced paricalcitol-induced monocytic differentiation in THP-1 monocytic leukemia cells.

U937 cells expressing PML-RARA (PR9) or vector control (B41) were treated with paricalcitol and arsenic trioxide. When B41 cells were cultured with paricalcitol and/or arsenic trioxide with or without zinc, or PR9 was cultured with with paricalcitol and/or arsenic trioxide without zinc, the results were similar to wild type U937 cells (FIG. 10C). In other words, paricalcitol induced monocytic differentiation and paricalcitol in the combination with arsenic trioxide enhanced the monocytic differentiation in these U937-derived cells without expressing PML-RARA (FIG. 10C). When PR9 cells were cultured with zinc, they expressed PML-RARα, as shown by western blot analysis, and differentiation induced by paricalcitol (0.01 μM) was partially blocked compared to PR9 without zinc. When arsenic trioxide was added to the medium, it decreased the protein level of PML-RARa (120 kb) induced by zinc in a dose dependent manner. As is shown in FIG. 10B, when the concentration of arsenic trioxide was over 0.4 μM, the protein expression was barely detectable in PR9 cells. The combination of paricalcitol and arsenic trioxide enhanced differentiation of PR9 cells in parallel with an arsenic trioxide-induced decrease of PML-RARα (FIG. 10C).

The mitchondrial enzyme, 1,25(OH)₂D₃ 24-hydroxylase (24-hydroxylase) is the target gene of vitamin D. It was shown that the transcriptional activity of this enzyme was activated by paricalcitol and enhanced by arsenic trioxide (FIG. 10A). This enzyme catalyzes the initial step in the conversion of the active molecule 1,25(OH)₂D₃ into less active metabolite, 24.25(OH)₂D₃ resulting in the inhibition of anti-proliferative effects of vitamin D.

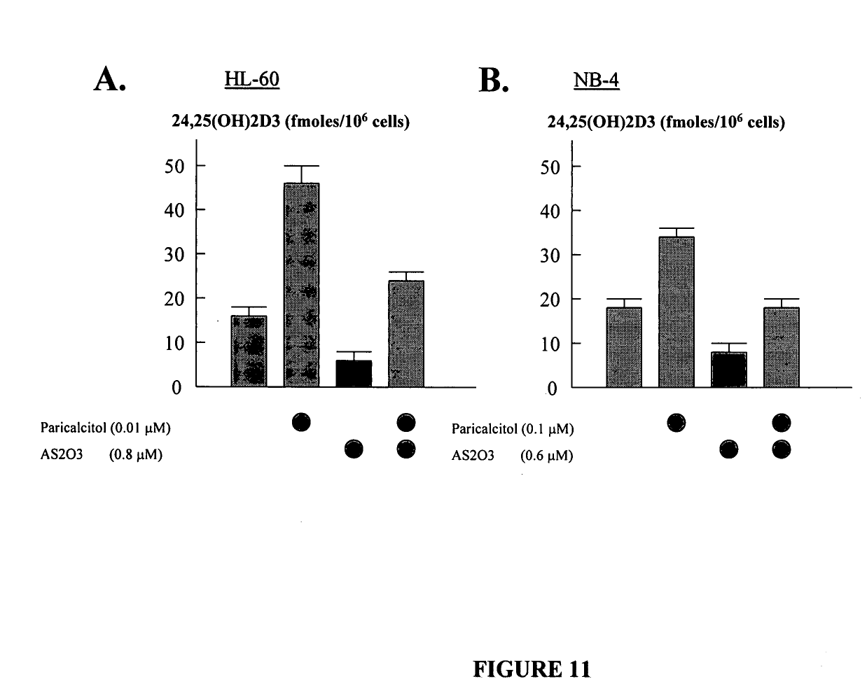

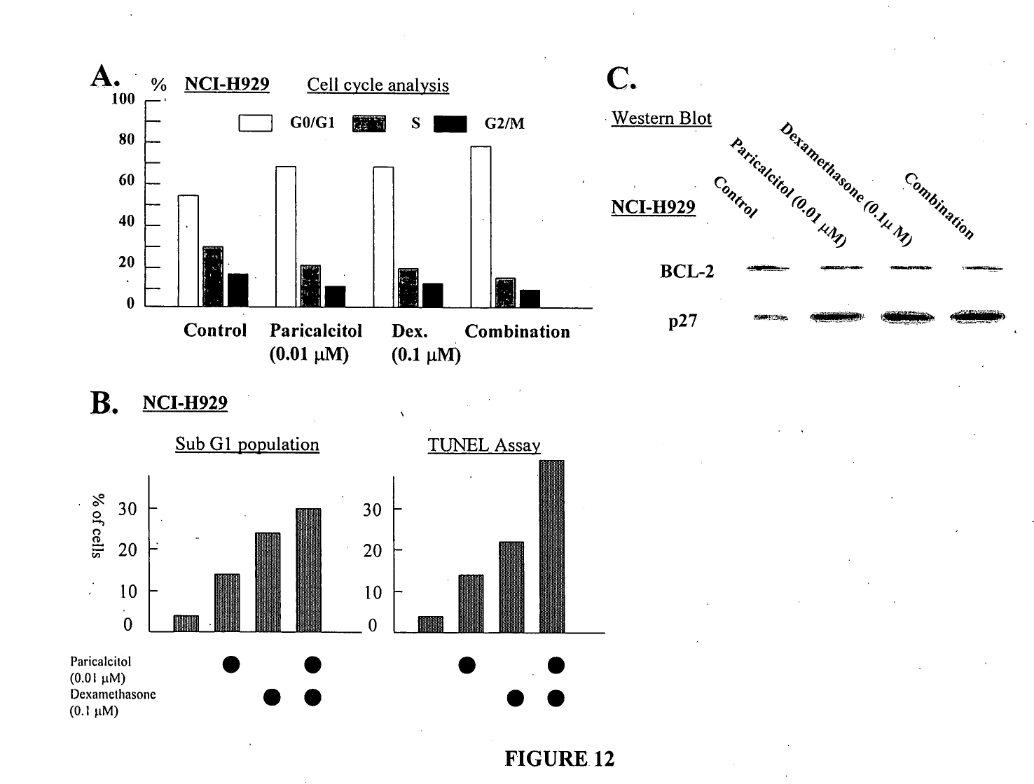

The possibility that the arsenic trioxide inactivates the mitchondrial enzyme, 24-hydroxylase leading to an activation of the response of the cells to vitamin D was tested. The activity of 24-hydroxylase was examined by measuring its metabolite, 24.25(OH)₂D₃ by TLC analysis in leukemia cells. HL-60 and NB-4 myeloid leukemia cell lines were treated with paricalcitol (0.01 μM for HL-60, 0.1 μM for NB-4) and/or arsenic trioxide (0.8 μM for HL-60, 0.6 μM for NB-4) for 3 days. Control cells were treated with vehicle alone. Then the levels of 24.25(OH)₂D₃ were measured by TLC analysis. Paricalcitol alone increased the levels of 24.25(OH)₂D₃ in HL-60 and NB-4, while arsenic trioxide alone decreased its level compared to control cells (FIG. 11). When cells were treated with paricalcitol and arsenic trioxide, the increased level was much smaller than paricalcitol alone in HL-60 calls and there was no increase compared to control cells in NB-4 cells (FIG. 11). The decrease of the levels of 24-hydroxylase by arsenic trioxide may explain the enhanced activity of paricalcitol by arsenic trioxide in leukemia cells.

### EXAMPLE XI

### Combination of Paricalcitol and Dexamethasone Inhibits Myeloma Cell Proliferation

This example shows that paricalcitol in combination with dexamethasone had profound antiproliferative activity against myeloma cells in vitro.

In addition to the combination of paricalcitol and arsenic trioxide, it was determined that the combination of paricalcitol and dexamethasone had strong antiproiferative against myeloma cell lines, NCI-H929 and RPMI8226 by MTT assay (FIG. 7E).

Myeloma cell line NCI-H929 was treated with paricalcitol (0.01 μM) and/or dexamethasone (0.01 μM) for 3 days. Control cells were treated with vehicle alone. Cell cycle analysis was performed by flow cytometry (FIG. 11A). Paricalcitol alone or arsenic trioxide alone induced G0/G1 arrest of NCI-H929. The combination slightly increased the G0/G1 accumulation compared to each drug alone. Percentage of sub-G1 population was measured by flow cytometry (FIG. 11B, left side). Sub-G1 population was increased by paricalcitol alone and arsenic trioxide alone. The combination clearly increased the sub-G1 population indicating apoptosis. TUNEL assay was also performed after 3 days for the quantitive analysis of the apoptotic cells (FIG. 11B, right side). Apoptotic cells were increased by paricalcitol alone and arsenic trioxide alone. The combination clearly increased the apoptotic cells by TUNEL assay. After treatment for 3 days, cell lysates were harvested and used for Western blotting using antibodies against Bcl-2 and p27^{KIP1}. It was observed that expression of Bcl-2 and p27^{KIP1} were down-regulated by each drug or their combination and the expression levels were not changed, indicating that the levels of these genes does not explain enhanced activity of the combination (FIG. 10C).

Throughout this application various publications have been referenced within parentheses. The disclosures of these publications in their entireties are hereby incorporated by reference in this application in order to more fully describe the state of the art to which this invention pertains.

1. A method of reducing the severity of a proliferative disorder, comprising administering to an individual having the proliferative disorder an effective amount of paricalcitol, wherein the paricalcitol reduces cellular proliferation, with the proviso that the proliferative disorder is not prostate cancer or head and neck squamous cell carcinoma.

2. The method of claim 1, wherein the proliferative disorder is cancer.

3. The method of claim 1, wherein the proliferative disorder is a myelodysplastic syndrome.

4. The method of claim 2, wherein the cancer is leukemia.

5. The method of claim 4, wherein the leukemia is acute myelocytic leukemia.

6. The method of claim 4, wherein the leukemia is acute lymphocytic leukemia.

7. The method of claim 2, wherein the cancer is multiple myeloma.

8. The method of claim 2, wherein the cancer is breast cancer or colon cancer.

9. A method of reducing the severity of a proliferative disorder, comprising administering to an individual having the proliferative disorder an effective amount of paricalcitol and an anti-cancer agent, wherein the combination of paricalcitol and the anti-cancer agent reduces cell proliferation, with the proviso that the proliferative disorder is not prostate cancer or head and neck squamous cell carcinoma.

10. The method of claim 9, wherein the proliferative disorder is cancer.

11. The method of claim 10, wherein the cancer is selected from leukemia, multiple myeloma, breast cancer and colon cancer.

12. The method of claim 9, wherein the proliferative disorder is a myelodysplastic syndrome.

13. The method of claim 9, wherein the anti-cancer agent is selected from daunomycin, arsenic trioxide, adriamycin, PS341, dexamethasone, taxol, 5-fluoroceracil and methotrexate.

14. The method of claim 13, wherein the anti-cancer agent is arsenic trioxide.

15. the method of claim 14, wherein the proliferative disorder is leukemia.

16. The method of claim 15, wherein the leukemia is acute myelocytic leukemia.

17. The method of claim 15, wherein the leukemia is acute lymphocytic leukemia.

18. The method of claim 13, wherein the anti-cancer agent is dexamethasone.

19. The method of claim 18, wherein the proliferative disorder is multiple myeloma.

20. The method of claim 13, wherein the anti-cancer agent is daunomycin.

21. The method of claim 20, wherein the proliferative disorder is myeloid leukemia.

22. The method of claim 13, wherein the anti-cancer agent is PS341.

23. The method of claim 22, wherein the proliferative disorder is myeloma.

24. The method of claim 13, wherein the anti-cancer agent is taxol.

25. The method of claim 24, wherein the proliferative disorder is prostate cancer.

26. The method of claim 24, wherein the proliferative disorder is breast cancer.

27. The method of claim 13, wherein the anti-cancer agent is adriamycin.

28. The method of claim 27, wherein the proliferative disorder is breast cancer.

29. The method of claim 13, wherein the anti-cancer agent is 5-fluoroceracil.

30. The method of claim 29, wherein the proliferative disorder is colon cancer.

31. The method of claim 13, wherein the anti-cancer agent is methotrexate.

32. The method of claim 31, wherein the proliferative disorder is colon cancer.

33. A method of reducing cancer recurrence, comprising administering to an individual in cancer remission an effective amount of paricalcitol, wherein the paricalcitol reduces cancer cell proliferation.

34. The method of claim 33, wherein the individual is in remission from leukemia.

35. The method of claim 34, wherein the leukemia is acute myelocytic leukemia.

36. The method of claim 34, wherein the leukemia is acute lymphocytic leukemia.

37. The method of claim 33, wherein the individual is in remission from multiple myeloma.

38. The method of claim 33, wherein the individual is in remission from breast cancer or colon cancer.

39. A method of reducing cancer recurrence, comprising administering to an individual in cancer remission an effective amount of paricalcitol and an anti-cancer agent, wherein the combination of paricalcitol and the anti-cancer agent reduces cancer cell proliferation.

40. The method of claim 39, wherein the individual is in remission from a cancer selected from leukemia, multiple myeloma, breast cancer and colon cancer.

41. The method of claim 39, wherein the anti-cancer agent is selected from daunomycin, arsenic trioxide, adriamycin, PS341, dexamethasone, taxol, 5-fluoroceracil and methotrexate.

42. The method of claim 41, wherein the anti-cancer agent is arsenic trioxide.

43. The method of claim 42, wherein the individual is in remission from leukemia.

44. The method of claim 43, wherein the leukemia is acute myelocytic leukemia.

45. The method of claim 43, wherein the leukemia is acute lymphocytic leukemia.

46. The method of claim 41, wherein the anti-cancer agent is dexamethasone.

47. The method of claim 46, wherein the individual is in remission from multiple myeloma.

48. The method of claim 41, wherein the anti-cancer agent is daunomycin.

49. The method of claim 48, wherein the individual is in remission from myeloid leukemia.

50. The method of claim 41, wherein the anti-cancer agent is PS341.

51. The method of claim 50, wherein the individual is in remission from myeloma.

52. The method of claim 41, wherein the anti-cancer agent is taxol.

53. The method of claim 52, wherein the individual is in remission from prostate cancer.

54. The method of claim 52, wherein the individual is in remission from breast cancer.

55. The method of claim 41, wherein the anti-cancer agent is adriamycin.

56. The method of claim 56, wherein the individual is in remission from breast cancer.

57. The method of claim 41, wherein the anti-cancer agent is 5-fluoroceracil.

58. The method of claim 57, wherein the individual is in remission from colon cancer.

59. The method of claim 41, wherein the anti-cancer agent is methotrexate.

60. The method of claim 59, wherein the individual is in remission from colon cancer.
